(19) United States
(12) Patent Application Publication
(10) Pub. No.: US 2016/0213647 A1
(43) Pub. Date: Jul. 28, 2016

(54) COMPOSITIONS AND METHODS FOR INHIBITING VIRAL INFECTION

(71) Applicants: ARNO THERAPEUTICS, INC., Flemington, NJ (US); OHIO STATE INNOVATION FOUNDATION, Columbus, OH (US)

(72) Inventors: Larry Schlesinger, Powell, OH (US); Jacob Yount, Columbus, OH (US); Alexander Zukiwski, Clarksburg, MD (US); Stefan Proniuk, Austin, TX (US); María Jesús Tuñón, Leon (ES); Keivan Zandi, Kuala Lumpur (MY)

(21) Appl. No.: 15/008,306

(22) Filed: Jan. 27, 2016

Related U.S. Application Data
(60) Provisional application No. 62/108,960, filed on Jan. 28, 2015.
(60) Provisional application No. 62/118,766, filed on Feb. 20, 2015.
(60) Provisional application No. 62/159,129, filed on May 8, 2015.
(60) Provisional application No. 62/190,706, filed on Jul. 9, 2015.

(51) Int. Cl. A61K 31/415; A61K 45/06

(52) U.S. Cl. CPC A61K 31/415; A61K 45/06

(57) ABSTRACT

Compositions and methods for inhibiting viral protein production, viral infection, and viral replication in a host or host cells infected with a virus (e.g., influenza, Junin, Chikungunya, Dengue, HIV, RHDV) by administering AR-12 and AR-12 analogs are provided.

### PRIORITY CLAIM

This application claims priority to U.S. Provisional Patent Application No. 62/108,960, filed on Jan. 28, 2015, U.S. Provisional Patent Application No. 62/118,766, filed on Feb. 20, 2015, U.S. Provisional Patent Application No. 62/159,129, filed on May 8, 2015, and U.S. Provisional Patent Application No. 62/190,706, filed on Jul. 9, 2015. The above referenced applications are incorporated herein by reference as if restated in full. All patent and non-patent references cited herein are incorporated by reference in their entirety.

### BACKGROUND

Between 5 and 20% of the population is infected with influenza virus in any given year and this virus and its complications remains one of the top ten causes of human mortality in the US [1, 2]. Currently, vaccines are the most common measure for reduction of disease. However, antigenic shift caused by mutations in viral proteins can render even those with pre-existing adaptive immunity susceptible to infection. Likewise, vaccines offer little protection against emergent influenza viruses, creating the potential for pandemics such as the 1918 Spanish Influenza pandemic and the 2009 H1N1 pandemic [1]. In recent years, virulent H5N1 and H7N9 viruses have also caused small outbreaks, leading to concerns about widespread illness should these viruses evolve to be readily transmissible between humans [3-7]. Thus, influenza virus is an ever-present health concern, and general strategies of defense are needed.

Drugs currently used to treat influenza virus infection are limited to two classes: neuraminidase inhibitors and adamantanes [8]. Both classes target viral proteins. Neuraminidase inhibitors target the viral neuraminidase, which normally allows the virus to detach itself from the cell as a final step in virus replication. Thus neuraminidase inhibitors must be used early in infection to have a significant effect. Adamantanes, such as the drug amantadine, inhibit the viral M2 protein, which is an ion channel necessary for viral uncoating during cellular infection. Since influenza virus is highly prone to mutation, resistance to one or both of these drug classes has been observed, and currently circulating strains have evolved complete resistance to amantadine, making this drug no longer useful clinically [8]. Thus, new drugs are needed. Since mammalian cells are far less prone to mutation than viruses, one way to combat the problem of resistance is to utilize drugs that target host cell pathways that are essential for virus replication.

AR-12 is an orally bioavailable small molecule celecoxib derivative (previously known as OSU-03012) that was discovered at The Ohio State University. The compound was initially developed in the oncology setting and a phase I study demonstrated an acceptable safety profile with long term oral exposures up to 33 weeks. The AR-12 doses studied in the oncology study most likely substantially exceed the exposure needed in the infectious disease setting. AR-12 has been previously shown to exhibit anti-tumor, anti-fungal, and anti-bacterial activity. It is thought that AR-12 induces autophagy of cells harboring intracellular bacteria and has been demonstrated to have direct activity against certain bacteria. Supportive preclinical studies demonstrated that AR-12 has rapid blood brain barrier penetration and appreciable accumulation in tissues, exceeding the blood level concentrations several fold [1].

AR-12 has been shown to have multiple biological activities in cells including the induction of autophagy [9-16]. The various cellular targets proposed for AR-12 make it unclear a priori what effect it would have on influenza virus infection. Likewise, the effects of autophagy on influenza virus infection and replication are not clear. For example, the influenza virus M2 protein has been described to inhibit cellular autophagy, suggesting that this cellular process is detrimental to the virus [17]. However, other studies have conversely shown that influenza virus proteins induce cellular autophagy, and that autophagy is beneficial for virus replication [18, 19]. We therefore empirically examined the effect of AR-12 pretreatment on influenza virus infection of macrophages, which are a significant source of inflammatory cytokines, reactive oxygen species, and other factors that contribute to acute lung injury during influenza virus infection [20, 21].

In addition to influenza viruses, other viruses cause a significant amount of diseased in both humans and animals. For example, Junin, Dengue, human immunodeficiency virus (HIV), herpes, and Chikungunya virus have inflicted human populations with both long-term and short-term infections that can lead to death and long-term illness. Effective vaccines and treatments have been difficult to identify for many viral infections and have limited efficacy due to viral variants and development of resistance to current treatments.

Viral Classification

The Baltimore classification organizes viruses into groups based on criteria including nucleic acid (DNA or RNA), strandedness (single or double-stranded), sense, and method of replication as follows:

TABLE 1
Group I: dsDNA viruses (e.g. Adenoviruses, Herpesviruses, Poxviruses)
Group II: ssDNA viruses (+strand or “sense”) DNA
(e.g. Parvoviruses)
Group III: dsRNA viruses (e.g. Reoviruses)
Group IV: (+)ssRNA viruses (+strand or sense) RNA
(e.g. Picornaviruses, Togaviruses)
Group V: (−)ssRNA viruses (−strand or antisense) RNA
(e.g. Orthomyxoviruses, Rhabdoviruses)
Group VI: ssRNA-RT (reverse transcribed) viruses (+strand or sense)
RNA with DNA intermediate in life-cycle (e.g. Retroviruses)

The precise antiviral mechanism of action continues to be evaluated, but an established body of evidence demonstrates that AR-12 (OSU-03012) is an inducer of host effector cell autophagy thus potentiating the cellular immune response. AR-12 is known to inhibit GRP78 (also known as BiP, HSPA5, resulting in the up-regulation of PERK, which, in turn, induces the formation of autophagosomes and subsequently host cell autophagy. AR-12 also down regulates the chaperone proteins HSP70, HSP27 and HSP90. The utilization of or “highjacking” of host chaperone proteins is critical for viral replication, assembly and homeostasis. The down regulation of GRP78 results in an upregulation of PERK, which is known be an inducer of host cell autophagy, and thus the two mechanisms of action as outlined above may be related or dependent.

Role of Molecular Chaperones in Viral Replication

Unfolded Protein Response

The molecular or protein chaperones are essential effectors in protein quality control, and serve as the primary checkpoint to organize/assist in the proper folding of polypeptides into their three dimensional conformational structure, prevent misfolded proteins from denaturation and aggregation and can also function to direct terminally misfolded proteins into the proteolytic system for degradation. Xiao 2010 [61], illustrates the involvement of the protein chaperones in the folding, and quality control of the viral protein folding and the assembly of viral proteins and virons into functional conformations within a cell.

Viruses rely on the underlying host cell machinery for viable viral replication. Multiple viral pathogens have evolved mechanisms to utilize (e.g. enhancement of protein folding) and subvert the host protein quality control machinery such as the unfolded protein response (UPR) to produce massive amounts of viral proteins, thereby enabling the completion of the viral life cycle. In addition, recent studies indicate that some viruses encode for their own viral chaperone-like proteins thereby enhancing infectivity [61].

The modulation of the host cell protein quality control elements, including the UPR and protein assembly, are critical for viral replication. Proteins requiring post-translational modifications are processed in the endoplasmic reticulum (ER) and a number of cellular “stresses” can lead to dysfunction of the ER and result in a distortion of the unfolded polypeptide load/misfolded protein load and the cell's protein-folding capacity. Cells have an intrinsic quality control system to monitor protein folding involving both the ER and the Golgi apparatus. Unfolded or misfolded proteins are tagged for degradation or sent back through the folding cycle. Sustained accumulation of unfolded polypeptides or incorrectly folded proteins can trigger the UPR. The UPR is a signaling program mediated by three transmembrane ER receptors: (1) activating transcription factor 6 (ATF6), (2) inositol requiring kinase 1 (IRE1) and (3) double-stranded RNA-activated protein kinase (PKR)-like endoplasmic reticulum kinase (PERK). In mammalian cells, the highly conserved ER resident chaperone, immunoglobulin heavy chain binding protein (BiP) (also known as glucose-regulated protein 78 (GRP78)) and heat shock 70 kDa protein 5 (HSPA5) are involved in the folding and assembly of nascent proteins and work as a master control of the UPR, interacting with three mediators: PERK, ATF6 and IRE1. Reid 2014 [57], He 2006 [41], Lee 2005 [48].

The UPR performs three functions; adaptation, alarm and apoptosis. During the UPR adaptation phase, the cell attempts to reestablish folding homeostasis by inducing the expression of chaperones that enhance protein folding. At the same time, cellular translation is reduced to decrease the ER protein folding load and the degradation of unfolded proteins is increased. If these two steps are unsuccessful, the UPR induces a cellular alarm along with the induction of mitochondrial mediated apoptosis. Chakrabarti 2011 [27]. He 2006 [41] illustrates the modulation of the UPR during viral infections.

Human Immunodeficiency Virus (HIV) is a Group VI retrovirus, identified as the causative agent of Acquired Immunodeficiency Syndrome (AIDS), a disease which has proven extremely difficult to treat secondary to the development of resistance mechanisms to the antiviral drugs. HIV is a single-stranded RNA retrovirus classified as a lentivirus. Lentiviruses also include SIV (Simian Immunodeficiency Virus, FIV (Feline Immunodeficiency Virus), Visna, and CAEV (Caprian Arthritis Encephalitis Virus).

HIV infects macrophages and CD4+ T cells by binding to the CD4 (cluster of differentiation 4) is a glycoprotein and other receptors on the cell surface and fusing with the cell membrane to gain entry to the cell. Viral proteins which are required for the viral infection (e.g., reverse transcriptase, integrase, ribonuclease, and integrase) are transcribed and produced. The HIV RNA is reversed transcribed into double-stranded DNA which integrates into the host cell DNA. During active viral replication, the integrated DNA provirus is transcribed into mRNA which is transcribed into viral proteins and packaged into a new virus particle. New virus particles exit the cell and can then infect additional host cells or be passed on to another host. The reverse transcriptase process is highly error prone, resulting in mutations that can lead to the development of anti-viral drug resistance.

Current therapies include nucleoside analogs, reverse transcriptase inhibitors, protease inhibitors, fusion or entry inhibitors, and integrase inhibitors. However, long term use of these therapies may result in the development of viral resistance or selection of resistant HIV strains which reduces the effectiveness of the treatment and may require the use of combinations of drugs along with the accompanying side effects and potential for drug-drug interactions.

Rabbit haemorrhagic disease virus (RHDV) is a Group IV virus of the family Caliciviridae and a causative agent of rabbit haemorrhagic disease (RHD). Hepatic injury and damage plays a central pathogenic role in RHD and the histopathology of RHD liver injury is similar to viral infections referred to in general as viral hepatitis which cause fulminant hepatic failure in humans. The RHDV model fulfils many of the requirements of an animal model of fulminant hepatic failure. (Vallejo 2014)[64].

Dengue infection is a serious viral disease in the tropical and subtropical areas of the world. It accounts for at least 500,000 hospital admission annually and consumes massive hospital resources. Dengue is endemic in many tropical and sub-tropical parts of the world and is rapidly spreading to other countries where the mosquito vectors, Aedes aegypti and Aedes albopictus are found. This mosquito-borne disease is estimated to pose health threat to at least 2.5 billion people living in endemic regions of the tropical and subtropical regions. It is currently among one of the most rapidly spreading mosquito-borne diseases.

Dengue virus (DENV) is an enveloped virus and a member of Flaviviridae family with four distinct genotypes (DENV-1, DENV-2, DENV-3 and DENV-4). Currently, there is no specific anti-dengue medication available and the treatment procedure is only supportive treatment. In addition, there is no approved dengue vaccine available. Therefore, there is an urgent need to find an antiviral treatment for Dengue virus.

What is needed are broad spectrum anti-viral compounds that interfere with mechanisms essential to viral replication, self-assembly, and propagation “upstream” from processes specific to a particular virus. Such compounds would have a reduced risk of developing resistant viruses and result in fewer side effects.

### SUMMARY

Aspects described herein provide methods and composition for inhibiting and/or preventing viral infection with AR-12.

AR-12 is a biologically active molecule with various proposed cellular targets. We have discovered that AR-12 inhibits influenza virus infection of macrophages, which are an important cell type mediating acute lung injury in influenza virus infections. Our results show that AR-12 may be a beneficial drug for the prevention or treatment of disease caused by influenza virus and other viruses that utilize similar entry or replication strategies. In addition, we have discovered AR-12 has substantial anti-viral activity across a multiple viral pathogens including, but not limited to Lassa, HIV-1, HIV-2, Drug Resistant HIV, Dengue, type 5 adenovirus, measles, mumps, coxsackie, cytomegalovirus (CMV) and Chikungunya (ChikV).

The inhibition of host protein chaperones by AR-12 is believed to result in the broad spectrum antiviral activity of AR-12 and the ability to overcome certain antiviral drug resistance mechanisms. Without being bound by theory, the anti-viral effect of AR-12 is believed to result from an enhancement of autophagy along with a down regulation/inhibition of GRP78 and other protein chaperones.

Aspects disclosed herein provide methods of inhibiting viral protein production in a host infected with a virus by administering AR-12 to the host wherein viral protein production is reduced by at least about 50% compared to an untreated host.

Aspects disclosed herein also provide methods of reducing viral infection of host cells by administering AR-12 to the host cells wherein the percent of infected cells is reduced by at least about 50% compared to untreated cells.

Further aspects disclosed herein provide methods of inhibiting viral replication in a host infected with a virus by administering AR-12 to the host in an amount sufficient to reduce viral replication in the host by at least about 50%.

Aspects disclosed herein provide methods of reducing viral infection in a host infected with Chikungunya virus by administering AR-12 to the host in an amount sufficient to achieve a blood, tissue, or organ concentration from about 1 μM to 100 μM.

Aspects disclosed herein also provide methods of reducing viral infection in a host infected with Junin virus comprising administering AR-12 to the host in an amount sufficient to achieve a blood, tissue, or organ concentration of about 0.15 μM to 0.55 μM.

AR-12 (a.k.a. OSU-03012) has been previously shown to exhibit anti-tumor, anti-fungal, and anti-bacterial activity. It is thought that AR-12 induces autophagy of cells harboring intracellular bacteria. However, to date this mechanism of AR-12 activity has not been shown with respect to influenza and other viruses.

Further aspects described herein provide methods and composition for inhibiting and/or preventing retroviral replication and/or proliferation with AR-12. In one aspect, the retrovirus is a lentivirus. In another aspect, the lentivirus is selected from the group consisting of HIV-1, HIV-1 with drug resistance (DR), and HIV-2. In another aspect, AR-12 is provided to a host infected with an HIV DR strain in an amount sufficient to achieve a blood or tissue concentration of at least about 0.30 μM.

Additional aspects provide methods of increasing the survival rate of a host having viral-induced fulminant hepatic failure, comprising administering AR-12 to the host in an amount of at least about 25 mg/kg.

Further aspects provide methods of reducing Dengue virus infection in a host infected with Dengue virus by administering AR-12 to the host in an amount sufficient to achieve a blood, tissue, or organ concentration of at least about 1 μM.

### BRIEF DESCRIPTION OF THE DRAWINGS

The feature and nature of the present disclosure will become more apparent from the detailed description set forth below when taken in conjunction with the accompanying drawings.

FIG. 1 shows an exemplary Western blot showing that AR-12 inhibits the product of influence A virus proteins (nucleoprotein and matrix protein 1) in RAW264.7 cells;

FIG. 2 shows that AR-12 prevents influenza virus A infections of RAW264.7 cells;

FIG. 3 shows the exemplary effects of AR-12 on the expression of Influenza A H3N2 strain and cell viability in the HEK293T reporter cell line (ELVIRA®);

FIG. 4 shows the exemplary effects of AR-12 on the expression of Influenza A H1N1 strain and cell viability in the HEK293T reporter cell line (ELVIRA®);

FIG. 5 shows exemplary dose-response curves for cytotoxicity of Vero and A549 cells treated with AR-12;

FIG. 6 shows exemplary viral titer results in Vero (top panel) and A549 cells (bottom panel) in three strains of Junin virus after treatment with AR-12;

FIG. 7 shows the exemplary antiviral and antimetabolic effects of AR-12 in Vero A cells infected with Chikungunya Virus;

FIG. 8 shows the exemplary antiviral effects of AR-12 in Vero A cells infected with the wild type (WT) and T-705 (favipiravir) resistant (RES) strains of Chikungunya Virus;

FIG. 9 shows the results of an exemplary study of the inhibition of HIV-1 91US001 replication in PBMC cells by AR-12;

FIG. 10 shows the results of an exemplary study of the inhibition of HIV-1 92HT599 replication in PBMC cells by AR-12;

FIG. 11 shows the results of an exemplary study of the inhibition of HIV-2 CBL20 H9 replication in PBMC cells by AR-12;

FIG. 12 shows the results of an exemplary study of the inhibition of HIV-2 CDC 310319 replication in PBMC cells by AR-12;

FIG. 13 shows the results of an exemplary study of the inhibition of HIV-1 MDR769 replication in PBMC cells by AR-12;

FIG. 14 shows the results of an exemplary study of the inhibition of HIV-1 91US001 replication in PBMC cells by AZT;

FIG. 15 shows the results of an exemplary study of the inhibition of HIV-1 92HT599 replication in PBMC cells by AZT;

FIG. 16 shows the results of an exemplary study of the inhibition of HIV-2 CBL20 H9 replication in PBMC cells by AZT;

FIG. 17 shows the results of an exemplary study of the inhibition of HIV-2 CDC 310319 replication in PBMC cells by AZT;

FIG. 18 shows the results of an exemplary study of the inhibition of HIV-1 MDR769 replication in PBMC cells by AZT;

FIG. 19 shows the results of an exemplary study of the inhibition of HIV-1 MDR769 replication in PBMC cells by Efavirenz;

FIG. 20 shows the results of an exemplary study of the inhibition of HIV-1 MDR769 replication in PBMC cells by Lopinavir; and

FIG. 21 shows the results of an exemplary study of survival rates after infection with RHDV, and provides the percentage of surviving rabbits following infection with RHDV and treatment with AR-12 or vehicle;

FIG. 22 shows the results of an exemplary study of the effect of RHDV infection and AR-12 treatment on blood biochemistry; and

FIG. 23 shows the results of an exemplary study of the viral yield reduction for cells infected with Dengue virus.

### DETAILED DESCRIPTION

The disclosed methods, compositions, and devices below may be described both generally as well as specifically. It should be noted that when the description is specific to an aspect, that aspect should in no way limit the scope of the methods.

AR-12 has been proposed to target multiple cellular pathways. Targeting host pathways to inhibit a virus infection decreases the likelihood of drug resistance as mammalian cells are less prone to mutation during replication. Additionally, our experiments in which AR-12 inhibited influenza virus infection of macrophages utilized a strain that is amantadine-resistant. Our experiments in which AR-12 inhibited Chikungunya virus utilized a strain that is T-705 (favipiravir) resistant. Thus, AR-12 can be active against viral strains that are resistant to other therapeutics.

AR-12 has been shown to be an antibacterial agent, but antiviral activity has not yet previously been reported for this compound. Our results are the first to show inhibition of influenza virus infection by AR-12.

As used herein, the term “AR-12” refers to (C₂₆H₁₉F₃N₄O and 2-amino-N-(4-(5-(phenanthren-2-yl)-3-(trifluoromethyl)-1H-pyrazol-1-yl)phenyl)acetamide)) having the following structure:

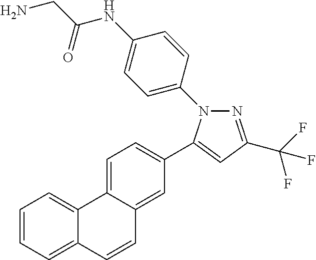 [SMILES: NCC(=O)Nc1ccc(-n2nc(C(F)(F)F)cc2-c2ccc3c(ccc4ccccc43)c2)cc1]

The term “AR-12” also includes salts and, for example, analogs of AR-12 (e.g., the compounds described in U.S. Pat. Nos. 7,576,116, 8,546,441, 8,541,460, 8,039,502, and 8,080,574 hereby incorporated by reference in their entirety)

Aspects described herein prove methods of inhibiting viral protein production in a host infected with a virus by administering AR-12 to the host wherein viral protein production is reduced by about 50% compared to an untreated host. As used herein, the term “administer” or “administered” refers to applying, ingesting, inhaling or injecting, or prescribing an active ingredient to treat a host or patient in need of treatment. The host can be a mammal (e.g., human, dog, cat, horse, or cow). An “untreated host” is a host that has not received AR-12 or another antiviral treatment.

In another aspect, the viral protein is produced by influenza or influenza A. In a further aspect, the viral protein is a structural or non-structural protein (e.g., matrix protein and nucleoprotein respectively).

Yet further aspects provide methods of reducing viral infection of host cells, including viral protein production, by administering AR-12 to the host cells wherein the percent of infected cells is reduced by about 50% compared to untreated cells. In this aspect, the viral protein is produced by influenza or influenza A. In addition, in this aspect, the viral protein can be a structural or non-structural protein (e.g., matrix protein or a nucleoprotein, respectively).

Another aspect provides methods of inhibiting viral replication in a host infected with a virus by administering AR-12 to the host in an amount sufficient to reduce viral replication in the host by about 50%. In this aspect, the virus is selected from the group consisting of a Group I virus, Group IV virus, Group V virus, and Group VII virus.

In this aspect, the virus can be selected from the group consisting of Influenza Strains (e.g., H3N2 and H1N1), Junin virus, Chikungunya virus, HIV (including drug resistant HIV), rabbit hemorrhagic fever virus (RHDV), Yellow Fever virus, Dengue virus, Pichinde virus, Measles virus, Punta Toro virus, Respiratory Syncytial virus, Rift Valley virus, SARS coronavirus, Tacaribe virus, and West Nile virus.

In one aspect, AR-12 is provided to the host in an amount sufficient to achieve a blood, tissue, or organ concentration from about 0.1 μM to about 7 μM.

In another aspect, AR-12 can be provided to a host infected with Chikungunya virus in an amount sufficient to achieve a concentration in the host cell of about 1 μM to 100 μM. In yet another aspect, the Chikungunya virus is a favipiravir-resistant strain of Chikungunya virus. In this aspect, AR-12 has been shown to have activity against replication of a CHIKV (Chikungunya Virus—Group IV) strain which is resistant to favipiravir (T-705) secondary to a unique mutation (K291R) in the viral RNA-dependent RNA polymerase. The lysine in the matched 291 position in the viral RNA-dependent RNA polymerase is highly conserved in Group IV (positive single strand RNA) viruses and confers the target of favipiravir (T-705). The mechanism of this resistance to favipiravir (T-705) and other viral RNA polymerase inhibitors in Group IV viruses does not confer resistance to AR-12.

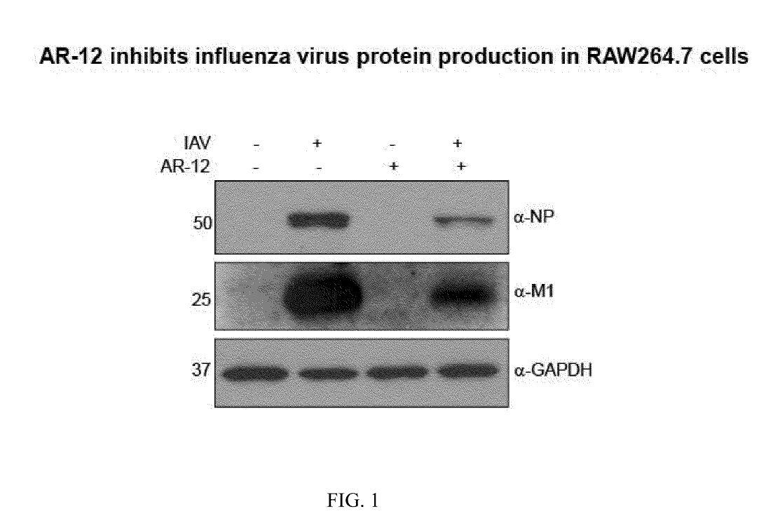

As shown in FIG. 1, AR-12 inhibits viral protein production. In this aspect, RAW264.7 cells were pretreated for 1 hour with 5 μM AR-12 or DMSO. Cells were infected with influenza A virus (IAV) strain PR8 (H1N1) in media+/−drug, harvested 24 hours post infection, and lysed for Western blotting. In addition, in this aspect, viral protein production is reduced by at least about 50%. The western blot shows levels of nucleoprotein (α-NP), matrix protein (α-M1) and α-GAPDH (Glyceraldehyde 3-phosphate dehydrogenase—control).

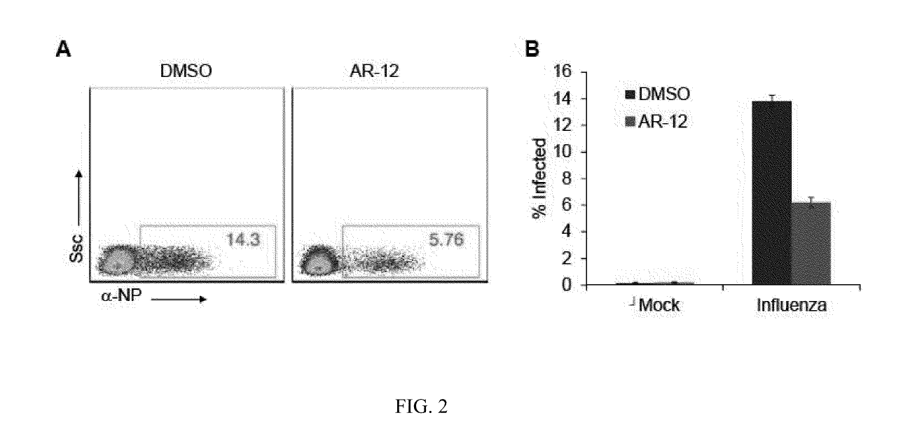

As shown in FIG. 2, AR-12 prevents viral infections. In this aspect, RAW264.7 cells were pretreated for 1 hour with 5 uM AR-12 or DMSO, infected with influenza A virus (IAV) strain PR8 (H1N1) in media+/−drugs, harvested 24 hours post infection, fixed with paraformaldehyde, and stained for influenza virus NP expression. Cells were analyzed by flow cytometry to determine % infection. Panel “A” shows representative flow cytometry data for infected cells. Panel “B” is a representative quantification of triplicate samples+/−standard deviation.

Table 2 below provides the results of an exemplary in vitro study measuring the EC50 and EC90 of AR-12 with respect to the indicated viruses.

TABLE 2

| | | | EC50 | EC90 | CC50 | | |
|---|---|---|---|---|---|---|---|
| Family | Species | Group | μM | μM | μM | Si50 | Si90 |
| Herpesviridae | Human | Group I | >2.40 | >2.40 | 6.3 | <3 | <3 |
| | cytomegalovirus | (dsDNA) | | | | | |
| | Varicella-zoster virus | Group I | 1.15 | 1.68 | 8.6 | 7 | 5 |
| | | (dsDNA) | | | | | |
| | Human herpes virus-6 | Group I | >0.48 | >0.48 | 0.67 | <1 | <1 |
| | | (dsDNA) | | | | | |
| Flaviviridae | Hepatitis C virus | Group IV | 0.12 | 0.19 | 0.44 | 4 | 2 |
| | | ((+)ssRNA) | | | | | |
| Papovaviridae | BK virus | Group I | >2.40 | >2.40 | 5.5 | <2 | <2 |
| | | (dsDNA) | | | | | |
| Poxviridae | Vaccinia | Group I | >2.40 | >2.40 | 10.81 | <5 | <5 |
| | | (dsDNA) | | | | | |
| Paramyxoviridae | Respiratory syncytial | Group V | | | | | |
| | virus | ((−)ssRNA) | | | | | |
| Hepadnaviridae | Hepatitis B virus | Group VII | 6.65 | 56.8 | 6.15 | 1 | <1 |
| | | (dsDNA- | | | | | |
| | | RT) | | | | | |
| Rhabdoviridae | Rabies Virus | Group V | 2.61 | — | 4.56 | 2 | — |
| | | ((−)ssRNA) | | | | | |
| Caliciviridae | Norovirus | Group IV | 0.718 | 8.1 | 6.4 | 8.9 | 0.8 |
| | | ((+)ssRNA) | | | | | |

In another aspect, AR-12 can be used to treat or prevent the illness and/or disease caused by the viruses listed above.

In one aspect, AR-12 inhibits viral replication of Group VII viruses as described herein.

AR-12 can be administered to a host infected with a retrovirus (e.g., lentivirus) in an amount sufficient to achieve a blood, tissue, or organ concentration at which viral replication is inhibited.

Aspects described herein provide methods of inhibiting retroviral replication in a host infected with a retrovirus, comprising administering AR-12 to the host in an amount sufficient to achieve a blood, tissue, or organ concentration of about 0.1 μM to about 20 μM.

In one aspect, AR-12 is administered to the host in an amount sufficient to achieve a blood or tissue concentration of at least about 0.30 μM.

Further aspects comprise administering at least a second compound to the host (e.g., azidothymidine (AZT), Efavirenz, and Lopinavir).

Yet another aspect provides methods of inhibiting replication of HIV DR in a host infected with HIV DR by administering AR-12 to the host in an amount sufficient to achieve a blood, tissue, or organ concentration of at least about 0.30 μM.

The term “IC50” refers to the inhibitory concentration of AR-12 at which viral replication is inhibited by 50%. In one aspect, the IC50 for AR-12 is less than about 0.5 μM. In another aspect, the IC50 is greater than about 0.2 μM. In yet another aspect, the IC50 is about 0.24 μM, about 0.27 μM, about 0.37 μM, or about 0.31 μM.

The term “TC50” refers to the toxic concentration (the concentration at which 50% of the test cells are killed. The “TC50/IC50 ratio” (also known as the selective index) is a measure of the level of a drug which causes toxicity to 50% the cells being tested in the experiment divided by the level of a drug which 50% reduction in the ability of the virus to replicate. In one aspect, the TC50/IC50 ratio for AR-12 is less than about 30. In another aspect, the TC50/IC50 ratio for AR-12 is about 25.6, about 22.6, about 19.9, about 16.4, or about 12

Table 3 below provides the results of an exemplary in vitro study measuring the IC50, TC50, and providing the TC50/IC50 ratio for AR-12, AZT (azidothymidine), Efavirenz, and Lopinavir in Peripheral Blood Mononuclear Cells (PBMCs) infected with the indicated HIV isolate.

TABLE 3

| | | | | | | Antiviral |
|---|---|---|---|---|---|---|
| Test | | High Test | | | | Index |
| Article | HIV Isolate | Concentration | | IC₅₀ | TC₅₀ | (TC₅₀/IC₅₀) |
| AR-12 | HIV-1 91US001 | 30 | μM | 0.24 | 6.12 | 25.6 |
| | HIV-1 92HT599 | | | 0.27 | | 22.6 |
| | HIV-2 CBL20 H9 | | | 0.51 | | 12.0 |
| | HIV-2 CDC 310319 | | | 0.37 | | 16.4 |
| | HIV-1 MDR 769 | | | 0.31 | | 19.9 |
| AZT | HIV-1 91US001 | 1,000 | nm | 17.4 | >1,000 | >57.5 |
| | HIV-1 92HT599 | | | 11.9 | | >84.0 |
| | HIV-2 CBL20 H9 | | | <0.10 | | >10,000 |
| | HIV-2 CDC 310319 | | | 5.73 | | >174 |
| | HIV-1 MDR 769 | | | >1,000 | | N/A |
| Efavirenz | | 1,000 | nm | 0.66 | >1,000 | >1,506 |
| Lopinavir | | 1,000 | nm | 827 | >1,000 | >1.21 |

N/A—Not Applicable

As shown in Table 3, the IC50 with respect to all tested HIV strains is in the micromolar range (e.g., 0.24 to 0.51 μM). The antiviral index of >10 provides a good margin of potential effectiveness versus tolerability.

In another aspect, AR-12 can be used alone or in combination with other anti-viral drugs including protease inhibitors (e.g., tipranavir, indinavir, atazanavir, saquinavir, lopinavir, ritonavir, darunavir, atazanavir, nelfinavir), nucleoside/nucleotide reverse transcriptase inhibitors (e.g., emtricitabine, lamivudine {3TC}, zidovudine {AZT}, didanosine, tenofovir, stavudine, abacavir), non-nucleoside reverse transcriptase inhibitors (e.g., rilpivirine, etravirine, nevirapine, delavirdine, efavirenz), entry inhibitors/fusion inhibitors (T-20, maraviroc), and integrase inhibitors (raltegravir, dolutegravir) to treat or prevent the illness and/or disease caused by HIV-1, drug resistant HIV-1 and HIV-2. Targeting those viral replication processes that are dependent on host biology allows for the potential to bypass resistance mechanisms secondary to mutations in the viral genome (e.g. mutated viral RNA polymerase) or prevent resistance from developing as the host protein chaperone targets and unfolded protein response pathways are evolutionarily conserved.

Aspects described herein provide methods of increasing the survival rate of a host with viral-induced fulminant hepatic failure by administering AR-12 to the host in an amount of at least about 25 mg/kg. In another aspect, the 25 mg/kg dose is administered at least four times (e.g., 0, 24, 36, 48 hours post-infection). In this aspect, the survival rate of the host is increased by at least about 20%.

AR-12 described herein can be administered orally, parenterally (IV, IM, depot-IM, SQ, and depot-SQ), sublingually, intranasally, inhalation, intrathecally, topically, by an ophthalmic route, or rectally. Dosage forms known to those of skill in the art are suitable for delivery of AR-12 described herein.

AR-12 can be formulated into suitable pharmaceutical preparations such as creams, gels, suspensions, ophthalmic preparations, tablets, capsules, inhalers, or elixirs for oral administration or in sterile solutions or suspensions for parenteral administration. AR-12 can be formulated into pharmaceutical compositions using techniques and procedures well known in the art.

In one aspect, about 0.1 to 1000 mg, about 5 to about 200 mg, or about 10 to about 50 mg of the AR-12, or a physiologically acceptable salt or ester can be compounded with a physiologically acceptable vehicle, carrier, excipient, binder, preservative, pain reliever, stabilizer, flavor, etc., in a unit dosage form as called for by accepted pharmaceutical practice. The amount of active substance in compositions or preparations comprising AR-12 is such that a suitable dosage in the range indicated is obtained.

In another aspect, the compositions can be formulated in a unit dosage form, each dosage containing from about 1 to about 1000 mg, about 1 to about 500 mg, or about 10 to about 200 mg of the active ingredient. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

In one aspect, one or more of AR-12 is mixed with a suitable pharmaceutically acceptable carrier to form compositions. Upon mixing or addition of the compound(s), the resulting mixture may be a cream, gel, solution, suspension, emulsion, or the like. Liposomal suspensions may also be used as pharmaceutically acceptable carriers. These may be prepared according to methods known to those skilled in the art. The form of the resulting mixture depends upon a number of factors, including the intended mode of administration and the solubility of the compound in the selected carrier or vehicle. In one aspect, the effective concentration is sufficient for lessening or ameliorating at least one symptom of the disease, disorder, or condition treated and may be empirically determined.

Pharmaceutical carriers or vehicles suitable for administration of AR-12 described herein include any such carriers suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

In another aspect, if AR-12 exhibits insufficient solubility, methods for solubilizing may be used. Such methods are known and include, but are not limited to, using co-solvents such as dimethylsulfoxide (DMSO), using surfactants (e.g., TWEEN, poloxamer) and dissolution in aqueous sodium bicarbonate. Derivatives of the compounds, such as salts or prodrugs, may also be used in formulating effective pharmaceutical compositions.

The concentration of the compound is effective for delivery of an amount upon administration that lessens or ameliorates at least one symptom of the disorder for which the compound is administered. Typically, the compositions are formulated for single dosage administration.

In another aspect, AR-12 as described herein may be prepared with carriers that protect them against rapid elimination from the body, such as time-release formulations or coatings. Such carriers include controlled release formulations, such as, but not limited to, microencapsulated delivery systems. The active compound can be included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo model systems for the treated disorder.

In another aspect, AR-12 and compositions described herein can be enclosed in multiple or single dose containers. The enclosed compounds and compositions can be provided in kits, for example, including component parts that can be assembled for use. For example, AR-12 in lyophilized form and a suitable diluent may be provided as separated components for combination prior to use. A kit may include AR-12 and a second therapeutic agent for co-administration. AR-12 and second therapeutic agent may be provided as separate component parts. A kit may include a plurality of containers, each container holding one or more unit dose of AR-12 described herein. In one aspect, the containers can be adapted for the desired mode of administration, including, but not limited to suspensions, solutions, tablets, gel capsules, sustained-release capsules, and the like for oral administration; depot products, pre-filled syringes, ampoules, vials, and the like for parenteral administration; and patches, medipads, gels, suspensions, creams, and the like for topical administration.

The concentration of AR-12 in the pharmaceutical composition will depend on absorption, inactivation, and excretion rates of the active compound, the dosage schedule, and amount administered as well as other factors known to those of skill in the art.

In another aspect, the active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient (e.g., any suitable filler/bulking agent) such as microcrystalline cellulose, starch, or lactose; a disintegrating agent such as, but not limited to, alginic acid and corn starch; a lubricant such as, but not limited to, magnesium stearate; a glidant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed or co-administered with other active materials that do not impair the desired action, or with materials that supplement the desired action. AR-12 can be used, for example, in combination with an antibiotic, antiviral, antifungal, pain reliever, or cosmetic (e.g., anti-influenza drugs such as Oseltamivir (Tamiflu®) and zanamivir (Relenza®) or other suitable anti-HIV drugs).

In one aspect, solutions or suspensions used for parenteral, intradermal, subcutaneous, inhalation, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, alcohols, polyethylene glycol, glycerin, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include, but are not limited to, physiological saline, phosphate buffered saline (PBS), and solutions containing thickening and solubilizing agents such as glucose, polyethylene glycol, polypropylene glycol, ethanol, N-methylpyrrolidone, surfactants and mixtures thereof. Liposomal suspensions including tissue-targeted liposomes may also be suitable as pharmaceutically acceptable carriers. These may be prepared according to methods known in the art.

In another aspect, AR-12 may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

In yet another aspect, compounds employed in the methods of the disclosure may be administered enterally or parenterally. When administered orally, compounds employed in the methods of the disclosure can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, they can be of the sustained release type so that the compounds employed in the methods described herein need to be administered only once or twice daily.

The dosage forms can be administered to the patient 1, 2, 3, or 4 times daily. AR-12 as described herein can be administered either three or fewer times, or even once or twice daily, or every other day.

The terms “therapeutically effective amount” and “therapeutically effective period of time” are used to denote treatments at dosages and for periods of time effective to reduce viral infection, viral replication, and/or viral levels. As noted above, such administration can be parenteral, oral, sublingual, transdermal, topical, intranasal, or intrarectal. In one aspect, when administered systemically, the therapeutic composition can be administered at a sufficient dosage to attain a blood level of the compounds of from about 0.1 μM to about 20 μM. For localized administration, much lower concentrations than this can be effective, and much higher concentrations may be tolerated. One of skill in the art will appreciate that such therapeutic effect resulting in a lower effective concentration of AR-12 may vary considerably depending on the tissue, organ, or the particular animal or patient to be treated. It is also understood that while a patient may be started at one dose, that dose may be varied overtime as the patient's condition changes.

It should be apparent to one skilled in the art that the exact dosage and frequency of administration will depend on the particular compounds employed in the methods of the disclosure administered, the particular condition being treated, the severity of the condition being treated, the age, weight, general physical condition of the particular patient, and other medication the individual may be taking as is well known to administering physicians who are skilled in this art.

### EXAMPLES

Not every element described herein is required. Indeed, a person of skill in the art will find numerous additional uses of and variations to the methods described herein, which the inventors intend to be limited only by the claims. All references cited herein are incorporated by reference in their entirety. The work described in certain examples described herein was conducted, as directed by the inventors, by Southern Research Institute using federal funds from the Division of AIDS, National Institute of Allergy and Infectious Diseases, National Institutes of Health under contract HHSN272201400010I entitled “In Vitro Testing Resources for HIV Therapeutics and Topical Microbicides.

### Example 1

RAW264.7 cells were grown in Dulbecco's Modified Eagle Medium supplemented with 10% fetal bovine serum in a humidified incubator maintained at 37° C. and with 5% CO2 levels. One hour prior to infection with the indicated virus, cells were treated with 5 uM AR-12 that was diluted from a stock solution dissolved in DMSO into cellular growth medium, or were treated with an equal volume of DMSO in growth medium.

### Example 2

Influenza virus A/PR/8/34 (H1N1) stocks were grown in 10-day embryonated chicken eggs for 48 h at 37° C., and were titered in Madin Darby Canine Kidney cells. After, 1 hour pretreatment of cells with drug or DMSO, virus was added to cells at a multiplicity of infection of 5 in medium containing DMSO or AR-12. Infection was allowed to proceed at 37° C. for 24 hours prior to collection of cells for flow cytometry or western blotting.

### Example 3

For western blotting, cells were lysed with buffer containing 1% Brij97 and protease inhibitors. Antibodies against influenza virus nucleoprotein (known as NP) and matrix protein 1 (known as M1) were used for blotting to examine the level of these proteins produced in the infected cells. Antibodies against GAPDH were used to demonstrate comparable protein loading in the different lanes. For flow cytometry, cells were fixed with 4% paraformaldehyde, permeabilized with 0.1% Triton X-100 in PBS, and were stained with antibodies against influenza NP that were directly conjugated to the fluorescent dye Alexafluor-647. Cells were analyzed on a Beckton Dickinson FACSCanto II flow cytometer and raw data was analyzed using Flowjo software.

### Example 4

### Primary EBOV Antiviral Testing by Yield Reduction Assay

For the yield reduction assay, HepG2 cells maintained in Modified Eagle's medium (MEM) with 10% fetal bovine serum (FBS), and 1× GlutaMax are plated in 24-well plates. The confluent monolayer is infected with EBOV at a MOI of 0.1 for 1 hour at 37° C. with rocking every 15 minutes. Following infection, medium containing virus is removed and plates washed four times with PBS to remove residual virus. Following washing, AR-12 is added in the medium at 6 concentrations in duplicate and plates will be incubated at 37° C. for 72 hours. Medium is collected at 72 hours post-infection for assessment by real time RT-PCR to determine the EBOV genome equivalents compared to a viral RNA standard. Carbocyclic 3-deazaadenosine or favipiravir (T-705) is used as the positive control and a media only negative control (no virus) and virus only control (no compound) is included. Antiviral activity can be calculated (effective concentration; EC) from the reduction in EBOV RNA levels (EC50 and EC90 values determined). The compound cytotoxicity (CC50) can be determined in uninfected cells by Promega's CellTiter Glo or CellTiter96 (MTS). The Selectivity Index (SI50) can be calculated as CC50/EC50.

### Example 5

### Secondary EBOV Antiviral Testing by Yield Reduction Assay

A secondary assay can include assessment of EBOV yield reduction by standard plaque assay. Plaque assays are more laborious than real time RT-PCR. As a result plaque assays are not suitable for primary screening, but can be used as a secondary method to confirm the yield reduction results generated by the RT-PCR endpoint. The secondary assay can be performed in a manner similar to the primary assay described above; however, at the end of the assay the samples collected from the assay plates are assessed for EBOV titer by plaque assay instead of real time RT-PCR. Plaque assays can be completed using 90-100% confluent Vero E6 cells in 6-well plates. Samples for titration can be serially diluted 10-fold and 200 μL can be added to each well. Plates are incubated for 1 h at 37° C. with rocking every 15 min. A primary overlay containing 1×EMEM, 5% FBS, and 0.9% agarose can be added to each well. Plates are incubated at 37° C. for 4-7 days followed by a secondary overlay, which is identical to the primary overlay with the addition of 5% neutral red. PFU will be counted on day 7-10 post-infection.

### Example 6

### Influenza A Virus

In one aspect, an assay can be performed in a HEK293T reporter cell line (ELVIRA®) to evaluate the effects of AR-12 on influenza A virus (IAV) infection. These cells have been stably transfected with a firefly luciferase (ffLuc) expression cassette driven by Influenza A virus promoter. Upon infection with IAV, the IAV polymerase is expressed in cells and drives the expression of ffLuc. The activity of ffLuc directly corresponds to the extent of infection in the cell population. In this aspect, treatment of cells with AR-12 inhibits IAV infection in a dose dependent manner and leads to reduction in ffLuc activity as shown, for example, in FIG. 1 with respect to Influenza Strain H3N2 and in FIG. 2 with respect to Influenza Strain H1N1. The compound SRI35204 refers to AR-12.

TABLE 4
EC50/CC50 for Influenza Strains H3N2 and H1N1

| | Influenza Strain | | | |
|---|---|---|---|---|
| | A/Udorn/72 (H3N2) | | A/Ca/07/09 (H1N1) | |
| Compound ID | EC50 | CC50 | EC50 | CC50 |
| SRI-35204 (AR-12) | 0.33 μM | >10 μM | 0.37 μM | >10 μM |
| Ribavirin | 5.24 μM | >100 μM | 12.85 μM | >100 μM |

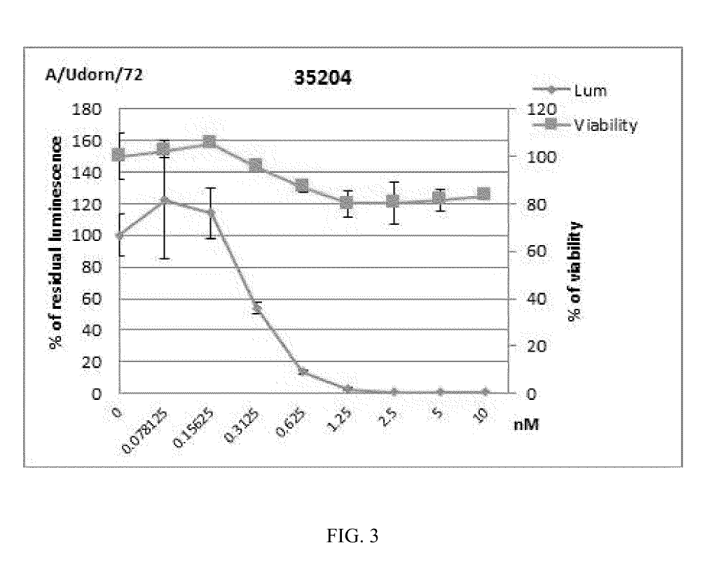

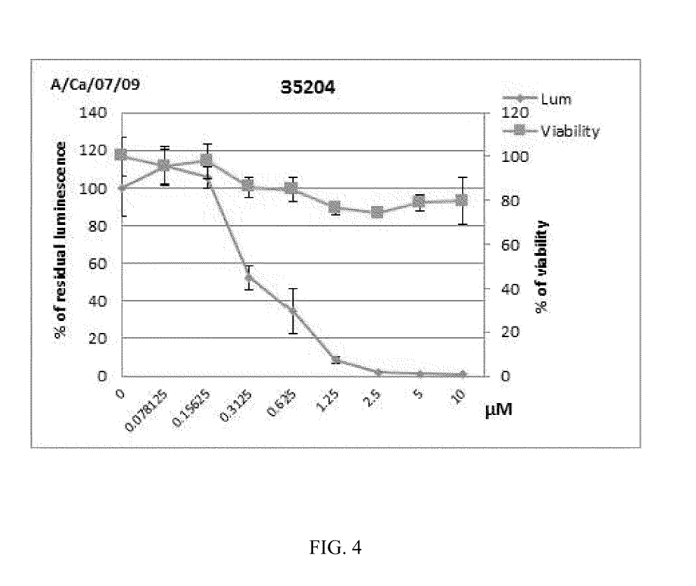

As shown in FIG. 3 and FIG. 4, the expression of the IAV polymerase (as measured by the percent of residual luminescence) decreases in a dose-dependent manner as the concentration of AR-12 increases from about 0.15 nm to about 1.25 nm in Influenza Strains H3N2 and H1N1 strains respectively. At concentrations than about 1.25 nm, the luminescence and percent cell viability approaches zero.

As shown in Table 3, the EC50 (half maximal effective concentration) for Influenza Strains H3N2 and H1N1 are 0.33 uM and 0.37 uM respectively. EC50 and CC50 (cytostatic concentration−the concentration required to reduce cell growth by 50%) values for AR-12 and Ribavirin are shown for comparison.

### Example 7

### Junin Virus

Cytotoxicity Assay

The cytotoxicity of AR-12 was tested in cultures of Vero and A549 cells by the colorimetric MTS method.

The cell lines Vero (African green monkey kidney) and A549 (lung carcinoma human cells) were grown in Eagle's minimum essential medium (MEM) supplemented with 5% fetal bovine serum. The serum concentration was reduced to 1.5% for maintenance medium (MM) in the biological assays.

AR-12 was dissolved in dimethyl-sulfoxide (DMSO) at 10 mM. Dilutions for cellular testing were further performed in MM. Cell viability was measured by the MTS [3-(4,5-dimethylthiazol-2-yl)-5-(3-carboxymethoxyphenyl)-2-(4-sulfophenyl)-2H-tetrazolium, inner salt; CellTiter 96® AQueous One Solution Reagent Promega] method. Confluent cultures of Vero and A549 cells in 96-well plates were exposed to serial concentrations of AR-12, using incubation conditions equivalent to those used in the antiviral assays (see below). 10 μl of MM containing MTS was added to each well. After 2 hours of incubation at 37° C., the absorbance was measured in a microplate reader at 490 nm. The cytotoxic concentration 50% (CC50) was calculated as the compound concentration necessary to reduce cell viability by 50%. All determinations were performed twice and each in triplicate.

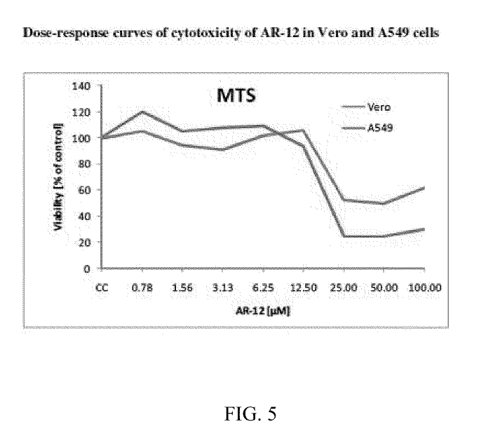

As shown in FIG. 5, the viability of both Vero and A549 cells in the presence of AR-12 was maintained at levels higher than 80-90% at concentrations lower than 12.5 μM. At higher concentrations, cell viability was reduced in a dose-dependent manner. From dose-response curves, the CC50s of AR-12 for Vero and A549 cells were 28.2±2.1 and 19.3±0.1 μM, respectively.

Antiviral Activity Assay

The antiviral activity of AR-12 was tested by virus yield inhibition assay in Vero and A549 cells.

In this example, the following attenuated virus strains of Junin virus (JUNV) were used: the vaccine strain Candid-1 [Maiztegui et al., J. Infect. Dis. 177, 277-83, 1998]; the avirulent XJC13 strain, derived from the prototype strain XJ [de Guerrero et al., Medicina (Bs.As.) 29, 1-5, 1969]; and the naturally attenuated IV4454 strain obtained from a mild human case [Contigiani et al., Medicina (Bs.As.) 37, 244-51, 1977]. The arenavirus Tacaribe (TCRV), strain TRLV11573 [Downs et al., Am. J. Trop. Med. Hyg. 12, 640-46, 1963], was also assayed.

For virus yield inhibition assays, confluent monolayers of Vero and A549 cells grown in 24-well plates were infected with each virus strain at a multiplicity of infection of 0.1 PFU/cell. After 1 hour adsorption at 37° C., cells were washed with MM and re-fed with MM containing serial non-cytotoxic concentrations of AR-12. After 48 hours of incubation at 37° C., supernatant cultures were harvested and extracellular virus yields were determined by a plaque assay in Vero cells. The effective concentration 50% (EC50) was calculated from dose-response curves as the compound concentration required to reduce the virus yield by 50% in the compound-treated cultures compared with untreated ones.

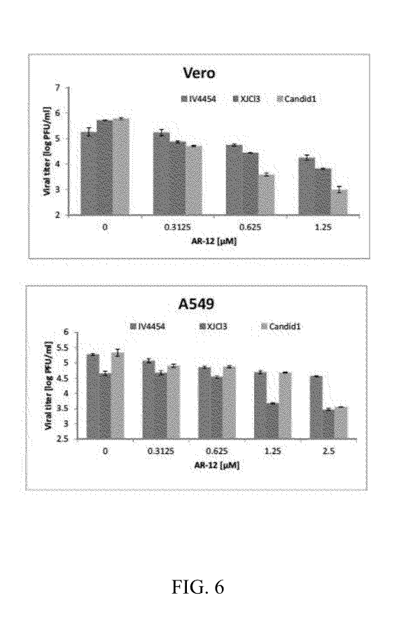

As shown in FIG. 6, the replication of the three JUNV strains was inhibited by AR-12 in a concentration-dependent manner. The EC50 values, calculated from the dose-response curves were in the range 0.15-0.55 μM, depending on the virus strain and the host cell, and the selectivity indices (ratio CC50/EC50) varied between 35.1 and 128.7. The activity against TCRV, antigenically close-related to JUNV was also tested. The EC50 values for TCRV in Vero and A549 cells were 0.35±0.03 and 0.84±0.02 μM, respectively, and the corresponding selectivity indices were 80.6 and 23.0.

TABLE 5
Anti-viral activity of AR-12 against Junin Virus

| | |
|---|---|
| Vero cells | A549 cells |

| | EC₅₀ | | EC₅₀ | |
|---|---|---|---|---|
| JUNV strain | (μM)^{a} | SI^{b} | (μM) | SI |
| XJCl3 | 0.23 ± 0.01 | 122.6 | 0.55 ± 0.05 | 35.1 |
| IV4454 | 0.51 ± 0.05 | 55.5 | 0.55 ± 0.05 | 35.1 |
| Candid-1 | <0.15 | >188.7 | 0.15 ± 0.02 | 128.7 |

^{a}EC₅₀ (effective concentration 50%) concentration required to reduce virus yield by 50% at 48 h post-infection.
^{b}SI (selectivity index): ratio CC₅₀/EC₅₀. The CC₅₀ values of AR-12 were 28.2 and 19.3 μm for Vero and A549 cells, respectively.

### Example 8

### Chikungunya Virus

About 4 mg of compound was transferred to a glass vial and DMSO was added to obtain a stock solution with a final concentration of 10 mg/m. The DMSO stock was stored at −20° C. when not used. Before each experiment, the thawed DMSO stock was checked for the presence of precipitate and only used when no precipitate was visible.

On day 1, Vero A cells were seeded in 100 μl of assay medium [MEM Rega 3 medium (Cat. No 19993013; Invitrogen) supplemented with 2% of fetal calf serum (Gibco), 1% sodium bicarbonate (Gibco, 25080060) and 1% of L-glutamine (25030024)] in 96-well tissue culture plates (Becton Dickinson) at a density of 2.5×104 cells/well and were allowed to adhere overnight in an incubator at 37° C. and 5% CO₂.

On day 2, 2× serial dilutions of AR-12 and T-705 (dilution factor of 1-to-3) were prepared in the assay medium on top of the cells, immediately after which 100 μl of a 2× dilution of CHIKV 899 (wild-type or T-705-resistant) was added to each well (with exception of the cell control wells). The final starting concentrations of compound were 100 μM and 200 μM for AR-12 and T-705, respectively, and the final multiplicity of infection (MOI) was 0.001. To evaluate the cytotoxicity of the compounds, a similar protocol was followed, but assay medium instead of the virus inoculum was added to the cells. To prevent carry-over effects when setting up the antiviral assays, a tip change was performed between each dilution step. A tip change was not deemed necessary for the cytotoxicity experiment. After setup, the assay plates were returned to the incubator (37° C.) for 5 days, a time at which maximal cytopathic effect is observed in the untreated, infected control conditions.

For the quantification of the antiviral effect, the assay medium was aspirated at the time the untreated, infected controls showed close to 100% virus-induced cell death (or cytopathic effect, assessed by microscopic inspection), followed by addition of 100 μl of a 10% MTS/PMS (3-(4,5-dimethylthiazol-2-yl)-5-(3-carboxymethoxyphenyl)-2-(4-sulfophenyl)-2H-tetrazolium/phenazine methosulfate, Promega) solution in phenol redfree medium and incubation for 1.5 hours (37° C., 5% CO2, 95-99% relative humidity) until an optical density (OD value) in the range of 0.6-0.8 was obtained. Absorbance was measured at a wavelength of 498 nm (Infinite F50, Tecan) and converted to percentage of untreated controls.

Analysis of the raw data, quality control and calculation of the EC50 value was performed employing a custom-made data processing software package (Accelrys). The EC50 (value derived from the dose-response curve) represents the concentration of compound at which 50% inhibition of virus-induced cell death is observed. All the assay wells were checked microscopically for minor signs of a virus-induced cytopathic effect (CPE) or alterations to the cells caused by the compound. A compound is considered to match the hit selection criteria if, at least at one concentration, complete protection of the cells from any signs of virus-induced cytopathic effects is observed without major alterations to the host cell and monolayer morphology. The CC50 (value derived from the dose-response curve) represents the concentration of compound at which the compound reduces the overall host cell metabolism of uninfected cells by 50%). The selectivity index (SI=CC50/EC50) provides an indication of the therapeutic window of the compound in this viruscell-based assay). Except for the calculation of the standard deviation, no further statistical analysis was performed.

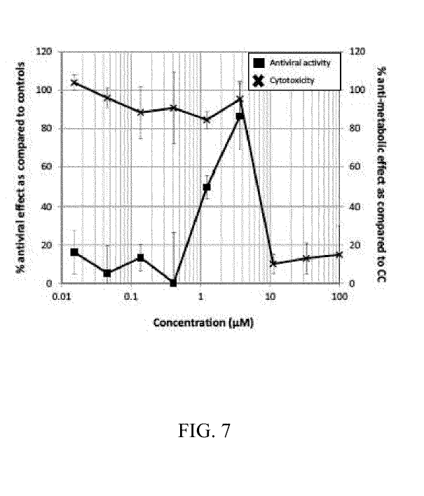

As shown in FIG. 7, for AR-12, at concentrations below 1 μM, no inhibition of virus-induced cell death (no antiviral activity, ▪ is observed), while the compound at these concentrations also does not significantly affect the host cell metabolism (no or only minor anti-metabolic effects are observed [symbol] ). At concentrations of AR-12 between 0.14 and 3.7 μM, a steep increase in cell survival is observed, which points towards a clear cell protective (antiviral) effect. At concentrations higher than 3.7 μM, the anti-metabolic effect of AR-12 is responsible for loss of overall metabolic activity of the cells (the anti-metabolic effect that is observed results from a combination of a direct anti-metabolic effect the compound may induce on host cell metabolism and/or cytostatic and/or cytotoxic effects).

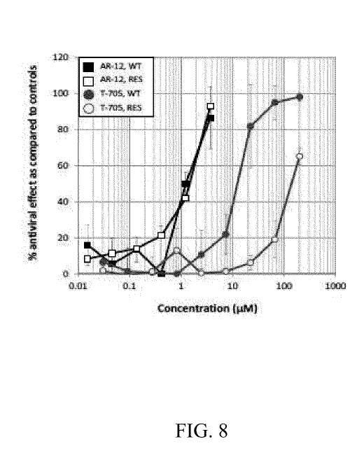

In a parallel experiment, the antiviral effect of AR-12 and T-705 was evaluated on the replication of wild-type and T-705-resistant chikungunya virus (FIG. 8).

TABLE 6
ChikV

| | EC₅₀ | EC₅₀ | |
|---|---|---|---|
| Compound | WT | T-705^{RES} | Fold resistance |
| Ar-12 | 1.1 ± 0.5 | 1.2 ± 0.8 | 1.1 |
| T-705 | 7.4 ± 3 | 138 ± 22 | 19 |

As expected, the antiviral activity of T-705 shifted to higher concentrations (from 7.4 to 138 μM, which is a decrease in sensitivity to the antiviral effect of T-705 with a factor 19). The antiviral activity of AR-12 remained unchanged.

When evaluated in cell culture, AR-12 inhibits chikungunya virus-induced cell death with an EC50 of 1.1±0.5 μM. In the same setting, the compound adversely affects the host cell metabolism with a CC50 of 6.6±0.3 μM, which, in this assay, results in a selectivity index of 5.8.

In a parallel experiment (FIG. 8) with T-705-resistant chikungunya virus, the antiviral potency of AR-12 is not affected. From this, it can be concluded that mutations the virus acquires to become less sensitive to the antiviral effect of T-705 do not affect the antiviral potency of AR-12. Taking into account the microscopic observations (FIGS. 7 and 8), the antiviral activity of AR-12 in this virus-cell-based assay is situated at the edge at which the adverse effects of the compound begin to dominate (in one assay, there is partial protection at one concentration and a cytotoxic effect at the next concentration, while in the other assay, there is full protection at the same concentration at which partial protection was observed in the previous assay).

### Example 9

### In Vitro Viral Screen

The results of an exemplary in vitro screen of the activity of AR-12 against various viruses is summarized below.

TABLE 7

| Virus | Cell Line | AR-12 Conc. | EC50 | EC90 | CC50 | Si50 | Si90 |
|---|---|---|---|---|---|---|---|
| Yellow Fever | Vero | 0.1-100 μg/ml | 0.32 (visual), | — | 2.8 (visual), 1.8 | 8.8 (visual), 6.4 | — |
| Virus - Strain | | | 0.28 Neutral | | (Neutral Red) | (Neutral Red) | |
| 17D | | | Red) | | | | |
| Dengue virus 2 - | Vero 76 | 0.1-100 μg/ml | 3.2 (visual), 0.86 | — | 3.2 (visual), 1.3 | 1.0 (visual), 1.5 | — |
| New Guinea C | | | (neutral red) | | (Neutral Red) | (Neutral Red) | |
| Pichinde Virus - | Vero | 0.1-100 μg/ml | 1.3 (visual), 2.2 | — | 3.2 (visual), 2.8 | 2.5 (visual), 1.3 | — |
| Strain An 4763 | | | (neutral red) | | (neutral red) | (neutral red) | |
| Measles Virus - | Vero 76 | 0.1-100 μg/ml | 2.6 (visual), 1.9 | — | 3.2 (visual), 3.4 | 1.2 (visual), 1.8 | — |
| Strain CC | | | (neutral red) | | (neutral red) | (neutral red) | |
| Punta Toro | Vero 76 | 0.1-100 μg/ml | 2.5 (visual), 1.0 | — | 2.8 (visual), 2.3 | 1.1 (visual), 2.3 | — |
| Virus - Adames | | | (neutral red) | | (neutral red) | (neutral red) | |
| Strain | | | | | | | |
| Respiratory | MA-104 | 0.1-100 μg/ml | 3.2 (visual), 3.1 | — | 3.2 (visual), 3.2 | 0 (visual), 1.0 | — |
| Syncytial Virus - | | | (neutral red) | | (neutral red) | (neutral red) | |
| Strain A2 | | | | | | | |
| Rift Valley | Vero 76 | 0.1-100 μg/ml | >100 (visual), >100 | — | >100 (visual), >100 | 0 (visual), 0 | — |
| Fever Virus - | | | (neutral | | (neutral red) | (neutral red) | |
| Strain MP-12 | | | red) | | | | |
| SARS | Vero 76 | 0.1-100 μg/ml | 3.2 (visual), >1.5 | — | 3.2 (visual), 1.5 | 0 (visual), 0 | — |
| coronavirus - | | | (neutral red) | | (neutral red) | (neutral red) | |
| Strain Urbani | | | | | | | |
| Tacaribe Virus - | Vero | 0.1-100 μg/ml | 0.62 (visual), | — | 2.8 (visual), 2.4 | 4.5 (visual), 2.8 | — |
| Strain TRVL | | | 0.86 (neutral red) | | (neutral red) | (neutral red) | |
| 11573 | | | | | | | |
| West Nile Virus - | Vero 76 | Infergen | 3.2 (visual), 2.6 | — | 3.2 (visual), 2.7 | 1.0 (visual), 1.0 | — |
| Strain Kerne | | | (neutral red) | | (neutral red) | (neutral red) | |
| 515, WN02 | | | | | | | |

FIG. 8 shows the exemplary antiviral effects of AR-12 in Vero A cells infected with the wild type (WT) and T-705 (favipiravir) resistant (RES) strains of Chikungunya Virus. As shown, AR-12 retains its antiviral effect against both wild-type and favipiravir resistant strains of Chikungunya virus in two cell types.

### Example 10

### Anti-HIV Efficacy Evaluation in Fresh Human PBMCs

Fresh human PBMCs, seronegative for HIV and HBV, are isolated from screened donors (Biological Specialty Corporation, Colmar, Pa.). Cells are pelleted/washed 2-3 times by low speed centrifugation and re-suspension in PBS to remove contaminating platelets. The Leukophoresed blood is then diluted 1:1 with Dulbecco's Phosphate Buffered Saline (DPBS) and layered over 14 mL of Lymphocyte Separation Medium (LSM; Cellgro® by Mediatech, Inc.; density 1.078+/−0.002 g/ml; Cat.#85-072-CL) in a 50 mL centrifuge tube and then centrifuged for 30 minutes at 600×g. Banded PBMCs are gently aspirated from the resulting interface and subsequently washed 2× with PBS by low speed centrifugation.

After the final wash, cells are enumerated by trypan blue exclusion and re-suspended at 1×106 cells/mL in RPMI 1640 supplemented with 15% Fetal Bovine Serum (FBS), and 2 mM L glutamine, 4 μg/mL Phytohemagglutinin (PHA, Sigma). The cells are allowed to incubate for 48-72 hours at 37° C. After incubation, PBMCs are centrifuged and re-suspended in RPMI 1640 with 15% FBS, 2 mM L-glutamine, 100 U/mL penicillin, 100 μg/mL streptomycin, and approximately 100-150 U/mL recombinant human IL-2 (R&D Systems, Inc). IL-2 is included in the culture medium to maintain the cell division initiated by the PHA mitogenic stimulation. PBMCs are maintained in this medium at a concentration of 1-2×106 cells/mL with biweekly medium changes until used in the assay protocol. Cells are kept in culture for a maximum of two weeks before being deemed too old for use in assays and discarded. MDMs are depleted from the culture as the result of adherence to the tissue culture flask.

For the standard PBMC assay, PHA stimulated cells from at least two normal donors are pooled (mixed together), diluted in fresh medium to a final concentration of 1×10⁶ cells/mL, and plated in the interior wells of a 96 well round bottom microplate at 50 μL/well (5×10⁴ cells/well) in a standard format developed by the Infectious Disease Research department of Southern Research Institute.

Pooling (mixing) of mononuclear cells from more than one donor is used to minimize the variability observed between individual donors, which results from quantitative and qualitative differences in HIV infection and overall response to the PHA and IL-2 of primary lymphocyte populations. Each plate contains virus/cell control wells (cells plus virus), experimental wells (drug plus cells plus virus) and compound control wells (drug plus media without cells, necessary for MTS monitoring of cytotoxicity). In this in vitro assay, PBMC viability remains high throughout the duration of the incubation period. Therefore, infected wells are used in the assessment of both antiviral activity and cytotoxicity. Test drug dilutions are prepared at a 2× concentration in microtiter tubes and 100 μL of each concentration (nine total concentrations) are placed in appropriate wells using the standard format. 50 μL of a predetermined dilution of virus stock is placed in each test well (final MOI 0.1).

The PBMC cultures are maintained for seven days following infection at 37° C., 5% CO₂. After this period, cell-free supernatant samples are collected for analysis of reverse transcriptase activity and/or p24 antigen content. Following removal of supernatant samples, compound cytotoxicity is measured by addition of MTS to the plates for determination of cell viability. Wells are also examined microscopically and any abnormalities are noted.

### Example 11

### Reverse Transcriptase Activity Assay

A microtiter plate-based reverse transcriptase (RT) reaction is utilized (Buckheit et al., AIDS Research and Human Retroviruses 7:295-302, 1991). Tritiated thymidine triphosphate (3H-TTP, 80 Ci/mmol, NEN) is received in 1:1 dH2O:Ethanol at 1 mCi/mL. Poly rA:oligo dT template:primer (GE Healthcare) is prepared as a stock solution by combining 150 μL poly rA (20 mg/mL) with 0.5 mL oligo dT (20 units/mL) and 5.35 mL sterile dH2O followed by aliquoting (1.0 mL) and storage at −20° C. The RT reaction buffer is prepared fresh on a daily basis and consists of 125 μL 1.0 M EGTA, 125 μL dH₂O, 125 μL 20% Triton X100, 50 μL 1.0 M Tris (pH 7.4), 50 μL 1.0 M DTT, and 40 μL 1.0 M MgCl2. The final reaction mixture is prepared by combining 1 part 3H-TTP, 4 parts dH₂O, 2.5 parts poly rA:oligo dT stock and 2.5 parts reaction buffer. Ten microliters of this reaction mixture is placed in a round bottom microtiter plate and 15 μL of virus containing supernatant is added and mixed. The plate is incubated at 37° C. for 60 minutes. Following incubation, the reaction volume is spotted onto DE81 filter-mats (Wallac), washed 5 times for 5 minutes each in a 5% sodium phosphate buffer or 2×SSC (Life Technologies). Next, they are washed 2 times for 1 minute each in distilled water, 2 times for 1 minute each in 70% ethanol, and then dried. Incorporated radioactivity (counts per minute, CPM) is quantified using standard liquid scintillation techniques.

### Example 12

### MTS Staining for PBMC Viability to Measure Cytotoxicity

At assay termination, assay plates are stained with the soluble tetrazolium-based dye MTS (CellTiter 96 Reagent, Promega) to determine cell viability and quantify compound toxicity. The mitochondrial enzymes of metabolically active cells metabolize MTS to yield a soluble formazan product. This allows the rapid quantitative analysis of cell viability and compound cytotoxicity. The MTS is a stable solution that does not require preparation before use. At termination of the assay, 20 μL of MTS reagent is added per well. The microtiter plates are then incubated 4-6 hrs at 37° C. The incubation intervals were chosen based on empirically determined times for optimal dye reduction. Adhesive plate sealers are used in place of the lids, the sealed plate is inverted several times to mix the soluble formazan product and the plate is read spectrophotometrically at 490/650 nm with a Molecular Devices

Vmax or SpectraMaxPlus Plate Reader.

Data Analysis

Using a computer program, IC50 (50% inhibition of virus replication), IC90 (90% inhibition of virus replication), IC95 (95% inhibition of virus replication), TC50 (50% cytotoxicity), TC90 (90% cytotoxicity), TC95 (95% cytotoxicity) and therapeutic index values (TI=TC/IC; also referred to as Antiviral Index or AI) are provided. Raw data for both antiviral activity and toxicity with a graphical representation of the data are provided in a printout summarizing the individual compound activity.

### Example 13

### Treatment of Rabbits Infected with RHDV

TABLE 9

| | | | | Frequency and | | Vol. of |
|---|---|---|---|---|---|---|
| | | Administration | | Administration | Dose | administration |
| Group | N | rate time | Treatment | Route | (mg/kg) | (ml/2 kg) |
| RHDV | 7 | 0, 12, 24, 36, | vehicle | Each 12 hours | — | ip 12.5 ml/kg |
| | | 48 hours pi | | ip | | ip 25 ml/rabbit |
| RHDV + | 7 | 0, 12, 24, 36, | AR-12 | Each 12 hours | 25 mg/ | ip 12.5 ml/kg |
| AR-12 | | 48 hours pi | | ip | Kg | ip 25 ml/rabbit |

Nine-week-old New Zealand white rabbits in a climatized room at 21° C., with a 12 h light cycle in accordance with the experimental design shown in Table 9 and provided standard dry rabbit food and water ad libitum. For survival studies, 14 animals were injected intramuscularly with 2×10⁴ hemagglutination units of RHDV isolate Ast/89 and six of them treated with AR-12 (25 mg/Kg body weight) at 0, 12, 24 and 36 hours post infection (50 mg/kg per day). Animals in those groups were left to die spontaneously.

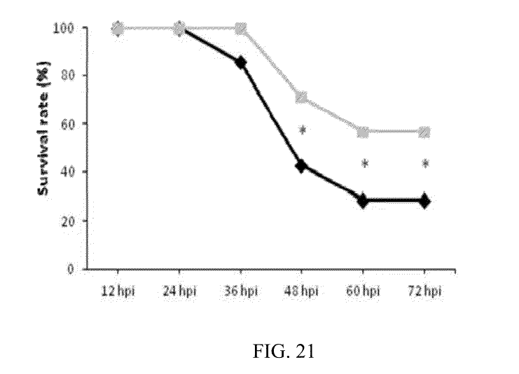

As shown in FIG. 21, the survival rates of infected rabbits increased significantly at least about 24 hours post infection (e.g., about 20%) (initial number n=7 per group. *p<0.05 with Fisher test).

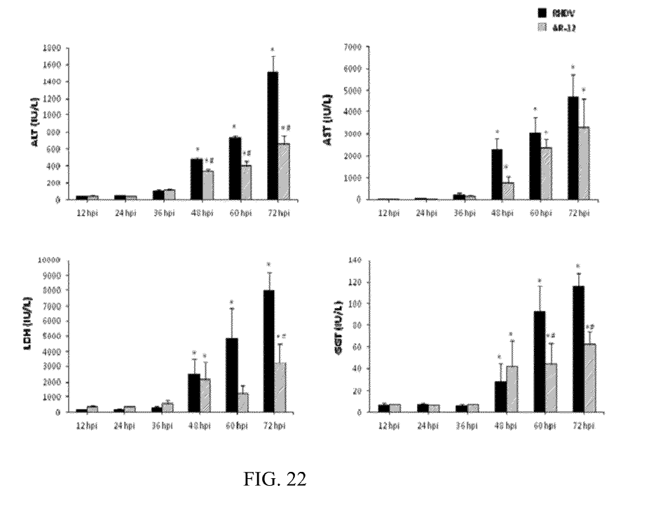

As shown in FIG. 22, AR-12 decreased the levels of markers of liver toxicity (ALT (Alanine transaminase), AST (Aspartate transaminase), LDH (lactate dehydrogenase), and GGT (gamma-glutamyl transferase) after about 36 hours post infection (*p<0.05 compared with 12 hpi. #p<0.05 compared with RHDV). These results indicate that AR-12 can treat, ameliorate, or reduce the symptoms associated with viral-induced fulminant hepatic failure.

### Example 14

### Anti-Viral Activity and Cytotoxicity of AR-12 in Standard PBMC Cell-Based Microtiter Anti-HIV Assay

The antiviral activity and cytotoxicity of AR-12 was tested in a standard PBMC cell-based microtiter anti-HIV assay against six (6) isolates representing different HIV drug resistance profiles.

AR-12 was supplied as a dry powder and was solubilized as described in Table 10. The stock was stored at 4° C. until the day of the assays. The compound stock solution was used to generate working drug dilutions used in the assays on each day of assay setup. Working solutions were made fresh for each experiment and were not stored for re-use in subsequent experiments performed on different days. The compounds were evaluated in the assays using a 10 μM (10,000 nM) high-test concentration with eight additional serial 1:2 dilutions (concentration range=39.1 nM to 10 μM).

Similarly, zidovudine (AZT; Nucleoside Reverse Transcriptase Inhibitor; NRTI) was included as a positive control antiviral compound using half-log dilutions and a concentration range from 100 pM to 1.0 μM (1,000 nM). Various additional controls were also included for assays depending on the resistance profile of the viruses. Nevirapine (Non-Nucleoside Reverse Transcriptase Inhibitor; NNRTI) and ritonavir (Protease Inhibitor; PI) were tested using a concentration range from 1.0 nM to 10 μM (10,000 nM). Delavirdine (NNRTI) and T-20 (Fusion Inhibitor) were tested using a concentration range from 200 pM to 2.0 μM (2,000 nM). Elvitegravir (Integrase Inhibitor; INT) was tested using a concentration range from 100 pM to 1.0 μM (1,000 nM). Finally, raltegravir (INI) was tested using a concentration range from 10 pM to 100 nM.

TABLE 10

| | Amount | Molecular | Concentration | | Compound | Data |
|---|---|---|---|---|---|---|
| Compound ID | (mg) | Weight | (mM) | Solvent | Color | Solubilized |
| AR-12 | 20.76 | 496.91 | 40 | DMSO | White/Off-white | Jun. 16, 2015 |

Efficacy Evaluation in Human Peripheral Blood Mononuclear Cells (PBMCs)

Virus Isolate Information

Six (6) HIV-1 isolates were selected for use in these experiments. These viruses include various drug resistant HIV-1 isolates to evaluate potential cross-resistance to AR-12. Unless otherwise noted, these virus isolates were obtained from the NIAID AIDS Research and Reference Reagent Program. Virus isolate 1022-48 was obtained from Dr. William A Schief of Merck Research Laboratories. MDR769 and MDR807 were obtained from Dr. Thomas C. Merigan of Stanford University. A low passage stock of each virus was prepared using fresh human PBMCs and stored in liquid nitrogen. Table 11 lists additional available information for each of the viruses. Pre-titered aliquots of the viruses were removed from the freezer and thawed rapidly to room temperature in a biological safety cabinet immediately before use.

TABLE 11

| | ENV | Co-receptor | |
|---|---|---|---|
| HIV-1 Isolate | Subtype | Tropism | Additional Information |
| NL4-3 | B | CXCR4 | HIV-1 molecular clone; wild-type control for NL4-3 |
| | | | based resistant isolates |
| MDR769 | B | CXCR4 | Multidrug resistant clinical isolate with drug resistance |
| | | | associated mutations in Protease (primary mutations |
| | | | M46L, I54V, V82A, I84V, & L90M) and Reverse |
| | | | Transcriptase (primary mutation M41L, K65R, |
| | | | Q151M, Y181I, &T215Y) |
| MDR807 | B | CXCR4 | Multidrug resistant clinical isolate with drug resistance |
| | | | associated mutations in Protease (primary mutations |
| | | | G48V, I54T, V82A) and Reverse Transcriptase (primary |
| | | | mutation M184V & T215Y) |
| A17 | B | CXCR4 | NNRTI-resistant variant of HIV-1 IIIB derived by |
| | | | passage in H9 cells in the presence of increasing |
| | | | concentration of a pyridinone NNRTI: contains K103N |
| | | | and Y181C mutations in the viral RT domain |
| 1022-48 | B | CXCR4 | PI resistant clinical isolate with Protease mutations |
| | | | L10I, T12S, I13V, L33I, M36I, M46I, I64V, V82F, |
| | | | I84V, L89M |
| 4736_4 | B | CXCR4 | INI resistant NL4-3 clone: HIB-1 plasma extracted from |
| | | | raltegravir treated individual and used to clone Integrase |
| | | | coding region into HIV-1 NL4-3; contains Integrase |
| | | | mutations E92Q & N155H |
| NL4-3 gp41 (36G) N42T, | B | CXCR4 | T-20 resistant NL4-3 clone with glycine at amino acid |
| N43K | | | position 36, and N42T and N43K mutations in gp41 |

Anti-HIV Efficacy Evaluation in Fresh Human PBMCs

Fresh human PBMCs were isolated from screened donors that were seronegative for HIV and HBV (Biological Specialty Corporation, Colmar, Pa.). Cells were pelleted/washed 2-3 times by low speed centrifugation and resuspension in Dulbecco's phosphate buffered saline (PBS) to remove contaminating platelets. The leukophoresed blood was then diluted 1:1 with PBS and layered over 14 mL of Ficoll-Hypaque density gradient (Lymphocyte Separation Medium, Cell Grow #85-072-CL, density 1.078+/−0.002 gm/mL) in a 50 mL centrifuge tube followed by centrifugation for 10 minutes at 600×g.

Banded PBMCs were gently aspirated from the resulting interface and subsequently washed 2× with PBS by low speed centrifugation. After the final wash, cells were enumerated by trypan blue exclusion and re-suspended at 1×10⁶ cells/mL in RPMI 1640 supplemented with 15% Fetal Bovine Serum (FBS), 2 mM L-glutamine, 100 U/mL penicillin, 100 μg/mL streptomycin, and 4 μg/mL Phytohemagglutinin (PHA; Sigma, St. Louis, Mo.; catalog #L1668). The cells were allowed to incubate for 48-72 hours at 37° C.

After incubation, PBMCs were centrifuged and resuspended in RPMI 1640 with 15% FBS, L-glutamine, penicillin, streptomycin, non-essential amino acids (MEM/NEAA; Hyclone; catalog #SH3023.01), and 20 U/mL recombinant human IL-2 (R&D Systems, Inc., Minneapolis, Minn.: catalog #202IL). PBMCs were maintained in this medium at a concentration of 1-2×10⁶ cells/mL, with twice-weekly medium changes until they were used in the assay protocol. Monocytes-derived-macrophages were depleted from the culture as the result of adherence to the tissue culture flask.

For the standard PBMC assay, PHA stimulated cells from at least two normal donors were pooled (mixed together), diluted in fresh medium to a final concentration of 1×10⁶ cells/mL, and plated in the interior wells of a 96 well round bottom microplate at 100 μL/well (5×10⁴ cells/well) in a standard format developed by the Infectious Disease Research department of Southern Research Institute. Pooling (mixing) of mononuclear cells from more than one donor is used to minimize the variability observed between individual donors, which results from quantitative and qualitative differences in HIV infection and overall response to the PHA and IL-2 of primary lymphocyte populations. Each plate contains virus control wells (cells plus virus) and experimental wells (drug plus cells plus virus). Test drug dilutions were prepared at a 4× concentration in microtiter tubes and 50 μL of each concentration was placed in appropriate wells using the standard format. Fifty (50) μL of a predetermined dilution of virus stock was placed in each test well (final MOI≅0.1). Separate plates were prepared identically without virus for drug cytotoxicity studies using an MTS assay system (described below; cytotoxicity plates also include compound control wells containing drug plus media without cells to control for colored compounds that affect the MTS assay). The PBMC cultures were maintained at 37° C., 5% CO₂ for six days following infection. After this period, cell-free supernatant samples were collected for analysis of reverse transcriptase activity and compound cytotoxicity was measured by addition of MTS to the separate cytotoxicity plates for determination of cell viability. Wells were also examined microscopically and any abnormalities were noted.

Reverse Transcriptase Activity Assay

A microtiter plate-based reverse transcriptase (RT) reaction was utilized (Buckheit et al., AIDS Research and Human Retroviruses 7:295-302, 1991). Tritiated thymidine triphosphate (3H-TTP, 80 Ci/mmol, PerkinElmer) was received in 1:1 dH2O:Ethanol at 1 mCi/mL. Poly rA:oligo dT template:primer (GE HealthCare) was prepared as a stock solution by combining 150 μL poly rA (20 mg/mL) with 0.5 mL oligo dT (20 units/mL) and 5.35 mL sterile dH₂O followed by aliquoting (1.0 mL) and storage at −20° C. The RT reaction buffer was prepared fresh on a daily basis and consisted of 125 μL 1.0 M EGTA, 125 μL dH2O, 125 μL 20% Triton X100, 50 μL 1.0 M Tris (pH 7.4), 50 μL 1.0 M DTT, and 40 μL 1.0 M MgCl2. The final reaction mixture was prepared by combining 1 part 3H-TTP, 4 parts dH2O, 2.5 parts poly rA:oligo dT stock and 2.5 parts reaction buffer. Ten microliters of this reaction mixture was placed in a round bottom microtiter plate and 15 μL of virus-containing supernatant was added and mixed. The plate was incubated at 37° C. for 60 minutes. Following incubation, the reaction volume was spotted onto DE81 filter-mats (Wallac), washed 5 times for 5 minutes each in a 5% sodium phosphate buffer or 2×SSC (Life Technologies), 2 times for 1 minute each in distilled water, 2 times for 1 minute each in 70% ethanol, and then dried. Incorporated radioactivity (counts per minute, CPM) was quantified using standard liquid scintillation techniques.

MTS Staining for Cell Viability

At assay termination, uninfected assay plates were stained with the soluble tetrazolium-based dye MTS (CellTiter 96 Reagent, Promega) to determine cell viability and quantify compound toxicity. MTS is metabolized by the mitochondria enzymes of metabolically active cells to yield a soluble formazan product, allowing the rapid quantitative analysis of cell viability and compound cytotoxicity. This reagent is a stable, single solution that does not require preparation before use. At termination of the assay, 20-25 μL of MTS reagent was added per well and the microtiter plates were then incubated 4-6 hours at 37° C./5% CO₂ to assess cell viability. Adhesive plate sealers were used in place of the lids, the sealed plates were inverted several times to mix the soluble formazan product and the plates were read spectrophotometrically at 490/650 nm with a Molecular Devices SpectraMax i3 plate reader.

Data Analysis

Using an in-house computer program, the PBMC data analysis includes the calculation of IC50 (50% inhibition of virus replication), IC90 (90% inhibition of virus replication), IC95 (95% inhibition of virus replication), TC50 (50% cytotoxicity), TC90 (90% cytotoxicity), TC95 (95% cytotoxicity) and therapeutic index values (TI=TC/IC; also referred to as Antiviral Index or AI). Raw data for both antiviral activity and toxicity with a graphical representation of the data are provided in a printout summarizing the individual compound activity.

Results

The results from the testing of AR-12 against HIV-1 in PBMCs are summarized below in Table 12 and the Figures described below:

TABLE 12

| HIV-1 | Compound | IC₅₀ | IC₉₀ | TC₅₀ | TI |
|---|---|---|---|---|---|
| Isolate | ID | (nM) | (nM) | (nM) | (TC₅₀/TC₅₀) |
| NL4-3 | AK-12 | 2,169 | 749 | 5,532 | 7.39 |
| | AZT | 39.6 | 9.96 | >1,000 | >100 |
| | Ritonavir | 92.3 | 45.5 | >10,000 | >220 |
| | Raltegravir | 5.18 | 1.30 | >100 | >77.0 |
| | Nevirapine | 227 | 75.3 | >10,000 | >133 |
| | Delavirdine | 57.6 | 30.6 | >2,000 | >65.5 |
| | T-20 | 412 | 133 | >2,000 | >15.0 |
| | Elvitegravir | 1.25 | 0.52 | >1,000 | >1,935 |
| MDR769 | AR-12 | 3,819 | 848 | 5,532 | 6.53 |
| | AZT | >1,000 | >1,000 | >1,000 | N/A |
| | Ritonavir | >10,000 | >10,000 | >10,000 | N/A |
| | Raltegravir | 8.95 | 3.64 | >100 | 27.4 |
| MDR807 | AR-12 | 2,126 | 905 | 5,532 | 6.12 |
| | AZT | >1,000 | 162 | >1,000 | >6.17 |
| | Ritonavir | 5,938 | 1,281 | >10,000 | >7.81 |
| | Raltegravir | 2.16 | 0.46 | >100 | >219 |
| A17 | AR-12 | 6,637 | 914 | 5,532 | 6.05 |
| | AZT | 27.4 | 11.6 | >1,000 | >86.0 |
| | Nevirapine | >10,000 | >10,000 | >10,000 | N/A |
| | Delavirdine | >2,000 | >2,000 | >2,000 | N/A |
| 1022-48 | AR-12 | 3,679 | 489 | 5,532 | 11.3 |
| | AZT | >1,000 | >1,000 | >1,000 | N/A |
| | Nevirapine | 242 | 70.4 | >10,000 | 142 |
| | Ritonavir | >10,000 | >10,000 | >10,000 | N/A |
| 4736_4 | AR-12 | 2,245 | 762 | 5,532 | 7.26 |
| | AZT | 20.6 | 2.57 | >1,000 | >389 |
| 4736_4 | Raltegravir | >100 | 74.5 | >100 | >1.34 |
| | Elvitegravir | 251 | 81.9 | >1,000 | >12.2 |
| NL4-3 gp41 (36G) | AR-12 | 7,372 | 1,016 | 5,532 | 5.45 |
| V42T, N43K | AZT | 40.3 | 11.1 | >1,000 | >90.1 |
| | T-20 | 1,773 | 646 | >2,000 | >3.10 |

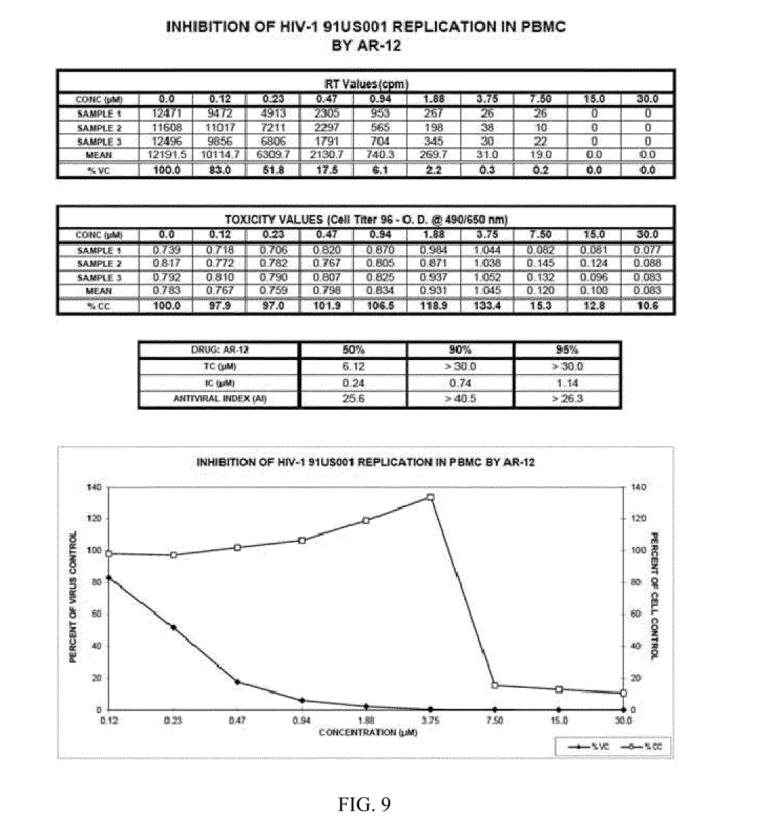

As shown in FIG. 9, the percent of virus control represents the reduction of viral replication as indicated by the black circles. In this example, an AR-12 concentration of 0.24 μM is required to achieve a 50% (TC50) inhibition of viral replication. The percent of cell control (e.g. cytotoxicity of AR-12) is indicated by the open squares. In this example, an AR-12 concentration of 6.12 μM required to achieve a 50% (CC50) toxicity of the PBMC.

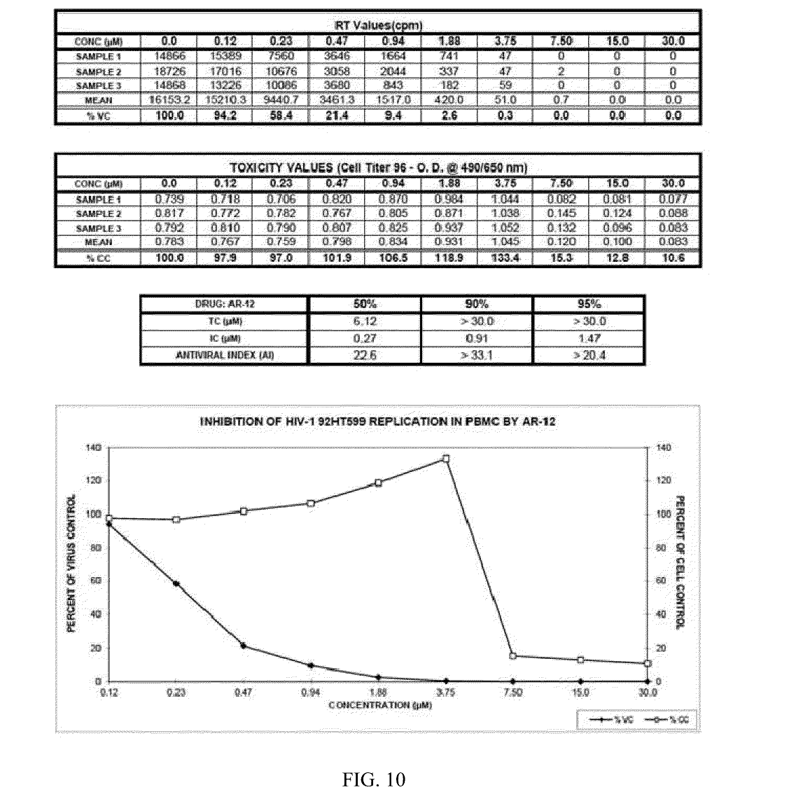

As shown in FIG. 10, the percent of virus control represents the reduction of viral replication and is indicated by the black circles. In this example, an AR-12 concentration of 0.27 μM is required to achieve a 50% (TC50) inhibition of viral replication. The percent of cell control (e.g. cytotoxicity of AR-12) is indicated by the open squares. In this example, an AR-12 concentration of 6.12 μM is required to achieve a 50% (CC50) toxicity of the PBMC.

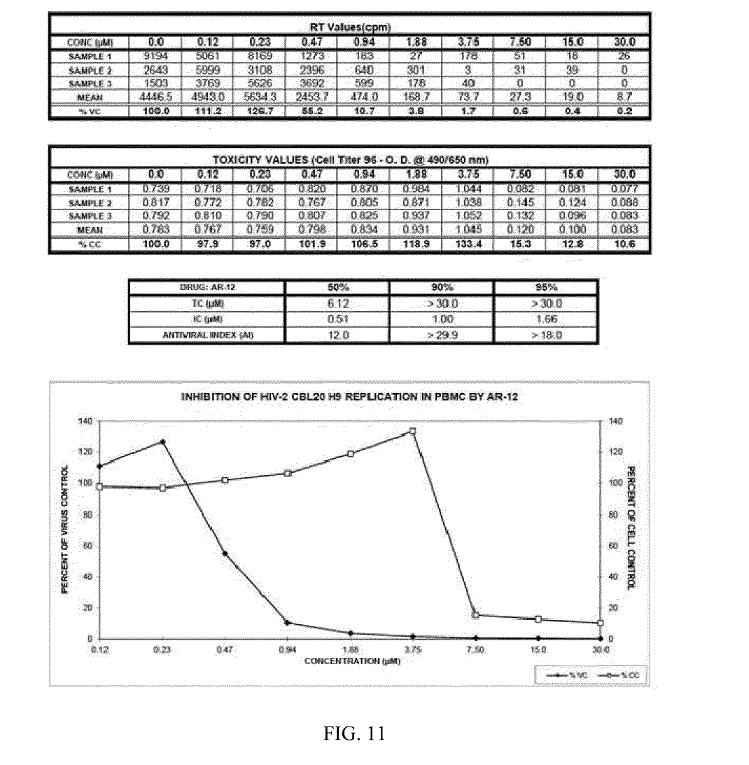

As shown in FIG. 11, the percent of virus control represents the reduction of viral replication and is indicated by the black circles. In this example, an AR-12 concentration of 0.51 μM is required to achieve a 50% (TC50) inhibition of viral replication. The percent of cell control (e.g. cytotoxicity of AR-12) is indicated by the open squares. In this example, an AR-12 concentration of 6.12 μM is required to achieve a 50% (CC50) toxicity of the PBMC.

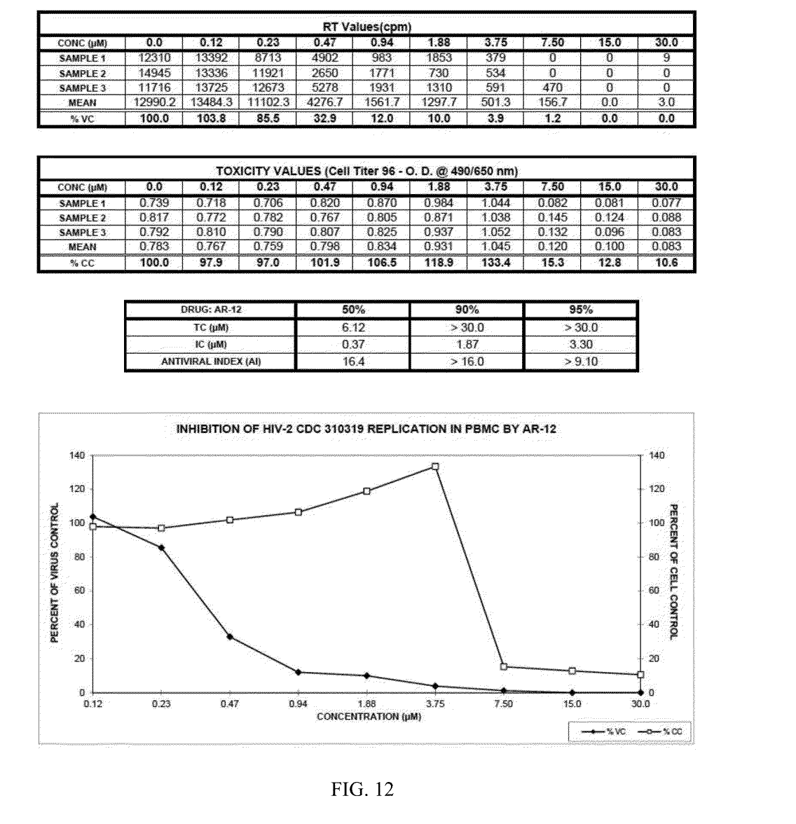

As shown in FIG. 12, the percent of virus control represents the reduction of viral replication and is indicated by the black circles. In this example, an AR-12 concentration of 0.37 μM is required to achieve a 50% (TC50) inhibition of viral replication. The percent of cell control (e.g. cytotoxicity of AR-12) is indicated by the open squares. In this example, an AR-12 concentration of 6.12 μM is required to achieve a 50% (CC50) toxicity of the PBMC.

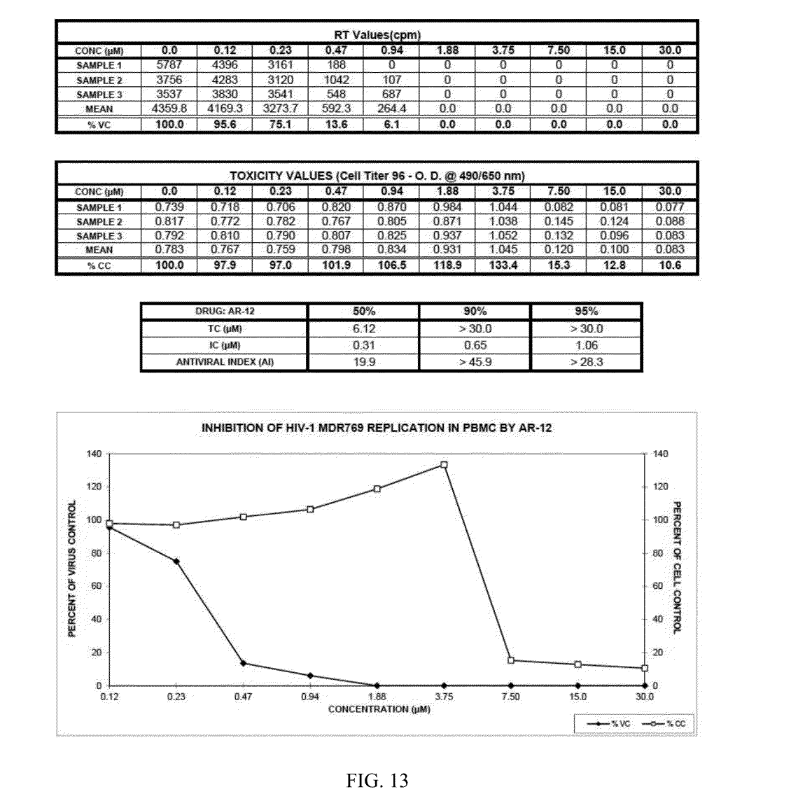

As shown in FIG. 13, the percent of virus control represents the reduction of viral replication and is indicated by the black circles. In this example, an AR-12 concentration of 0.31 μM required to achieve a 50% (TC50) inhibition of viral replication. The percent of cell control (e.g. cytotoxicity of AR-12) is indicated by the open squares. In this example, AR-12 concentration of 6.12 μM is required to achieve a 50% (CC50) toxicity of the PBMC.

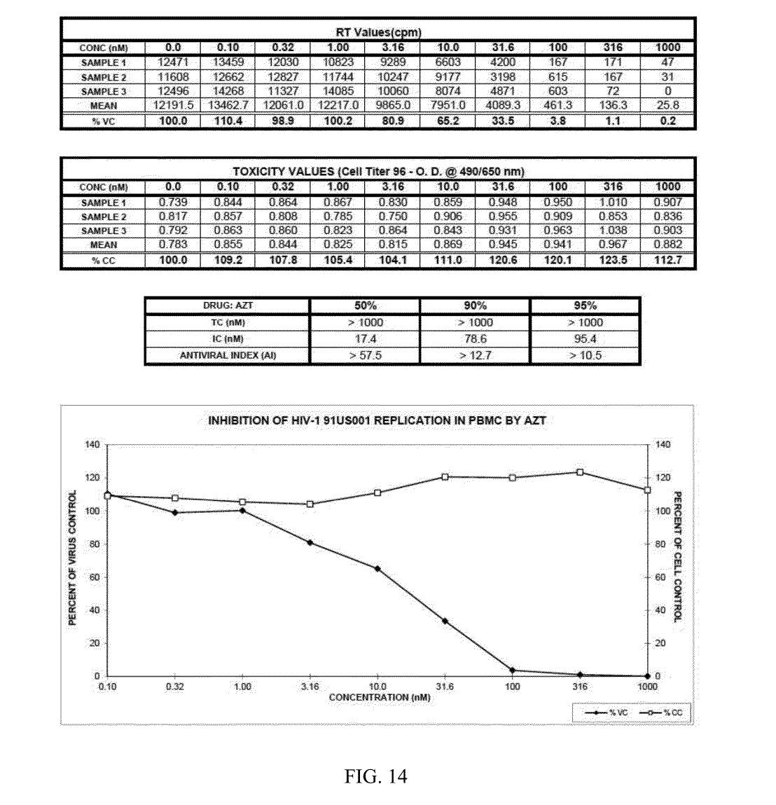

As shown in FIG. 14, the percent of virus control represents the reduction of viral replication and is indicated by the black circles. In this example, an AZT concentration of 17.4 nM is required to achieve a 50% (TC50) inhibition of viral replication. The percent of cell control (e.g. cytotoxicity of AZT) is indicated by the open squares. In this example, an AZT concentration of >1000 nM is required to achieve a 50% (CC50) toxicity of the PBMC.

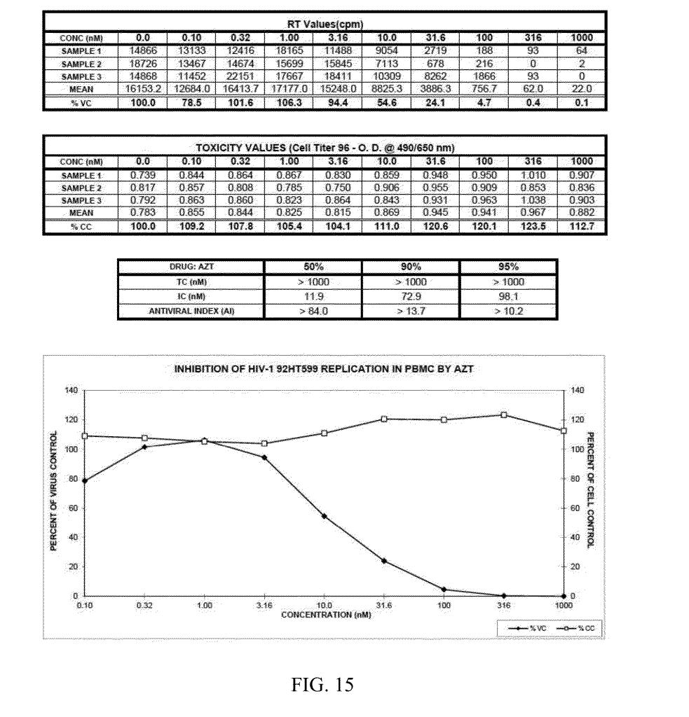

As shown in FIG. 15, the percent of virus control represents the reduction of viral replication and is indicated by the black circles. In this example, an AZT concentration of 11.9 nM required to result in a 50% (TC50) inhibition of viral replication. The percent of cell control (e.g. cytotoxicity of AZT) is indicated by the open squares. In this example, an AZT concentration of >1000 nM is required to achieve a 50% (CC50) toxicity of the PBMC.

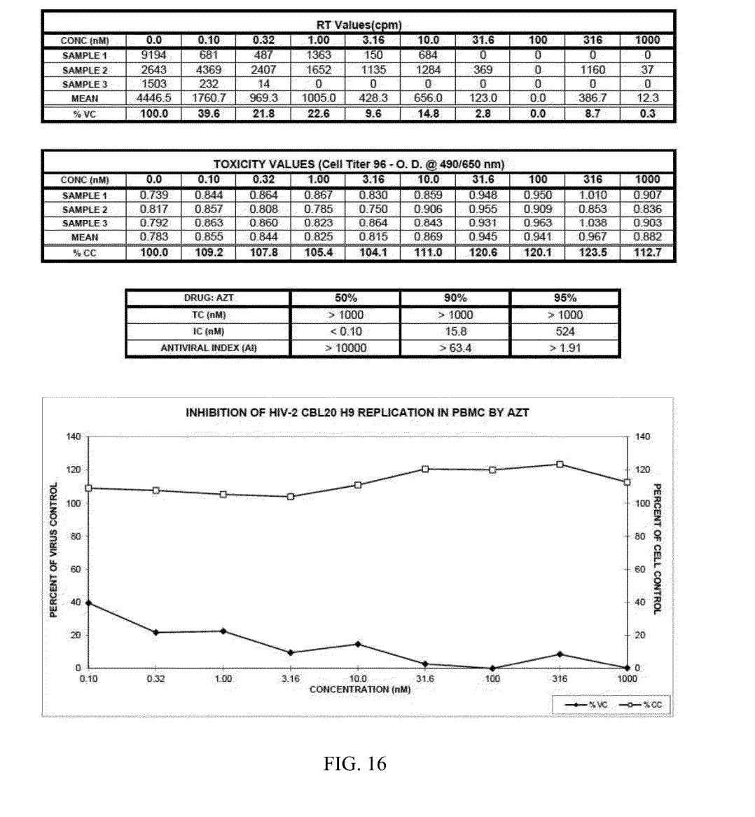

As shown in FIG. 16, the percent of virus control represents the reduction of viral replication and is indicated by the black circles with an AZT concentration of <0.10 nM required to result in a 50% (TC50) inhibition of viral replication. The percent of cell control (e.g. cytotoxicity of AZT) is indicated by the open squares. In this example, an AZT concentration of >1000 nM is required to achieve a 50% (CC50) toxicity of the PBMC.

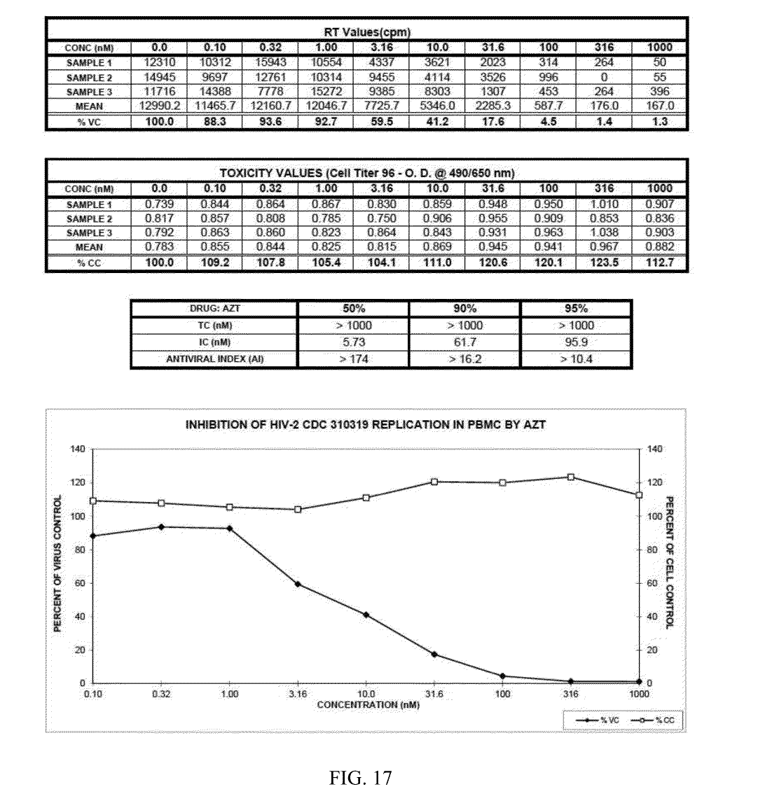

As shown in FIG. 17, the percent of virus control represents the reduction of viral replication and is indicated by the black circles. In this example, an AZT concentration of 5.73 nM is required to achieve a 50% (TC50) inhibition of viral replication. The percent of cell control (e.g. cytotoxicity of AZT) is indicated by the open squares. In this example, an AZT concentration of >1000 nM is required to achieve a 50% (CC50) toxicity of the PBMC.

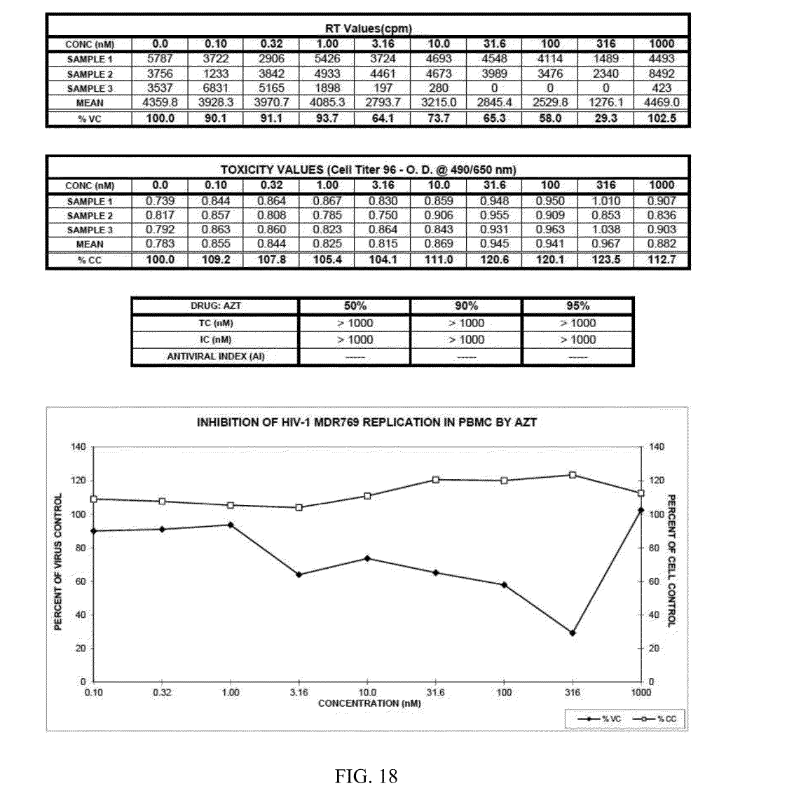

As shown in FIG. 18, the percent of virus control represents the reduction of viral replication and is indicated by the black circles. In this example, an AZT concentration of >1000 nM is required to achieve a 50% (TC50) inhibition of viral replication. The percent of cell control (e.g. cytotoxicity of AZT) is indicated by the open (squares), with an AZT concentration of >1000 nM required to result in a 50% (CC50) toxicity of the PBMC.

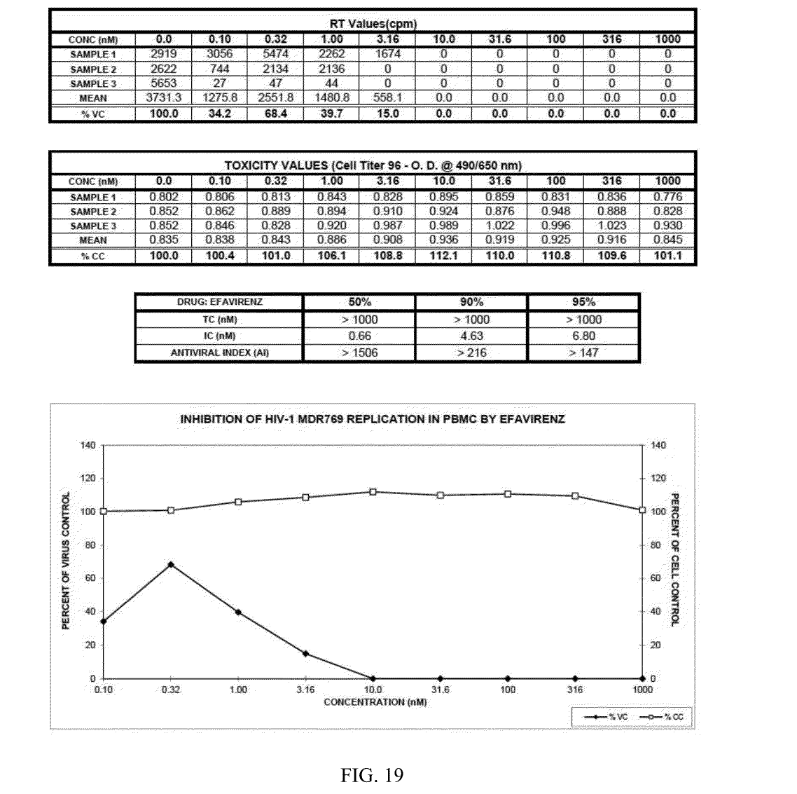

As shown in FIG. 19, the percent of virus control represents the reduction of viral replication and is indicated by the black circles. In this example, an efavirenz concentration of 0.66 nM is required to achieve a 50% (TC50) inhibition of viral replication. The percent of cell control (e.g. cytotoxicity of efavirenz) is indicated by the open squares. In this example, an efavirenz concentration of >1000 nM is required to achieve a 50% (CC50) toxicity of the PBMC.

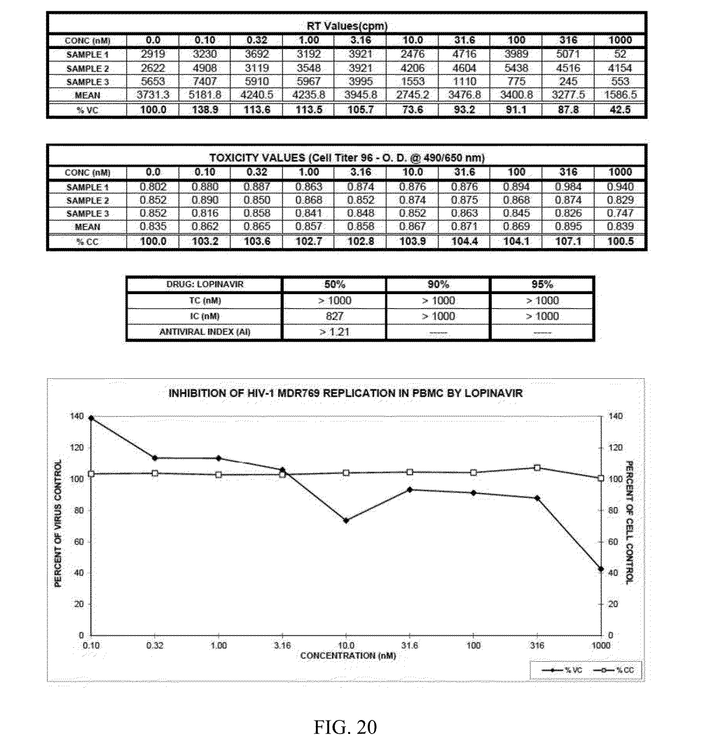

As shown in FIG. 20, the percent of virus control represents the reduction of viral replication and is indicated by the black circles. In this example, a lopinavir concentration of 877 nM is required to achieve a 50% (TC50) inhibition of viral replication. The percent of cell control (e.g. cytotoxicity of lopinavir) is indicated by the open squares. In this example, a lopinavir concentration of >1000 nM is required to achieve a 50% (CC50) toxicity of the PBMC.

AR-12 exhibited a dose-dependent reduction in virus replication for all virus isolates tested, with an average IC50 value of 812 nM (IC50 range=489 nM to 1,016 nM). Moderate cytotoxicity was observed (TC50=5,532 nM), resulting in average Therapeutic Index of 6.8 (TI range=5.45 to 11.3). There was no apparent resistance to AR-12 for any of the HIV drug resistant isolates evaluated in this study, suggesting that AR-12 inhibits HIV through a mechanism that is different from the NRTI, NNRTI, PI, INI and entry inhibitors to which these isolates are resistant.

The overall assay performances were validated by the control compounds (AZT, ritonavir, raltegravir, elvitegravir, nevirapine, delavirdine and T-20), which exhibited the expected levels of antiviral activity in each of the assays. Macroscopic observation of the cells in each well of the microtiter plates confirmed the cytotoxicity results obtained following staining of the cells with the MTS metabolic dye.

### Example 15

### Dengue Virus

DMSO was used to dissolve the lyophilized extract AR-12 and prepared stock solution was stored at −20° C. Stock solution was diluted using cell culture medium and sterilized by a syringe filter with 0.2 micron pore size (Millipore, MA, USA) right before each experiment.

Cells and Viruses

C6/36 mosquito cell line was used for the propagation of all DENV isolates used in the investigation. Vero cell line was used for the evaluation of antiviral activity. The cell lines were maintained and propagated in EMEM (Gibco, NY, USA) containing 10% fetal bovine serum (FBS, Gibco, NY, USA). The C6/36 and Vero cells were incubated at 28° C. and 37° C. in the presence of 3% and 5% CO2 respectively. At the time of virus inoculation and antiviral assays, the concentration of FBS was reduced to 2%. Four different clinical DENV isolates representing the four serotypes of DENV (DENV-1, DENV-2, DENV-3 and DENV-4) were used for analysis. The serotypes were identified in TIDREC (Tropical Infectious Diseases Research & Education Centre, Malaysia) from the patients' samples at University of Malaya Medical Center (UMMC). All four clinical isolates have been genotyped using full genome sequencing method. All the four clinical isolates propagated in C6/36 cell line. After titration of the virus isolates, stocks were stored at −80° C. until further use in the experiments.

In Vitro Cytotoxicity Assay

To determine the toxicity of AR-12 against Vero cells a MTS assay has been performed. Briefly, the confluent Vero cells in 96-well microplate were treated by different concentrations of AR-12 in triplicates. The treated cells were incubated for 2 days which was same to the time period for antiviral assay tests at 37° C. followed by the addition of 15 μl of MTS solution (Promega, WI, USA) to each well. The microplate was incubated at 37° C. for more 4 hours. Then, 100 μl of the solubilization/stopping solution was added to each well. The optical density (OD) of all wells including non-treated cells were read using 570 nm wavelength filter by plate reader (TECAN, Mannendorf, Switzerland). Cytotoxicity of the AR-12 was calculated using Graph Pad Prism 5 (Graph Pad Software Inc., San Diego, Calif.) along with its Dose-response curve plotting.

Antiviral Activity Assays

In order to determine the effects of AR-12 against DENVs in vitro, confluent monolayers of Vero cell line were infected with 200 FFU of each DENV serotypes and following virus adsorption for 1 hour at 37° C., the infected monolayer was rinsed twice with sterile PBS and supplemented with 2% FBS containing EMEM with different concentrations of AR-12. Later, the plates were incubated at 37° C. for 2 days in the presence of 5% CO2. DENVs yield has been evaluated by quantitative RT-PCR.

Quantitative RT-PCR

The antiviral effects of AR-12 has been confirmed by virus yield reduction assay using q RT-PCR by measuring the DENVs RNA copy number after 2 days post-treatment. Each one step qRT-PCR was carried out in a final volume of 20 μl containing 5 μl of diluted RNA, 1 μl of probe/primer mix, 10 μl of real time master mix and 4μ of nuclease-free water (PrimerDesign, Southampton, UK). Quantitative PCR measurement was performed using StepOnePlus real time PCR system (Applied Biosystems, USA) according to manufacturer's protocol. Raw data was analyzed with StepOne™ Software v2.2.1 to determine baseline and threshold for Ct. (Please see the Appendix A).

Statistical Analysis

The half maximal cytotoxicity concentration (CC50) and half maximal inhibitory concentration (IC50) were used as the main parameters in this investigation. GraphPad PRISM for Windows, version 5 (GraphPad Software Inc., San Diego, Calif., 2005) was used for all statistical analyses.

Results

Cytotoxic Activity of AR-12

Cytotoxicity of AR-12 on Vero cells were evaluated using the MTS assay. The MNTD was >10 μM. Therefore, we have chosen that concentration as the highest concentration for antiviral assay later.

Antiviral Assay of AR-12 Against DENVs

Virus yield reduction assay using a specific qRT-PCR for all 4 DENV serotypes has been used to evaluate the in vitro anti-dengue activity of AR-12 (FIG. 1).

AR-12 exhibited a dose-dependent inhibition effects against all 4 DENV genotypes replication in Vero cells with a half maximal inhibition concentration (IC50) values presented in As shown in Table 13 below, AR-12 has significant antiviral activity against different genotypes of DENV.

TABLE 13

| | Antiviral Activity IC₅₀ |
|---|---|
| Virus Serotype | (μM) |
| DENV-1 | 0.64 |
| DENV-2 | 0.8 |
| DENV-3 | 0.6 |
| DENV-4 | 0.89 |

Table 13. IC50 values of AR-12 against DENV serotypes. All results obtained from three independent experiments.

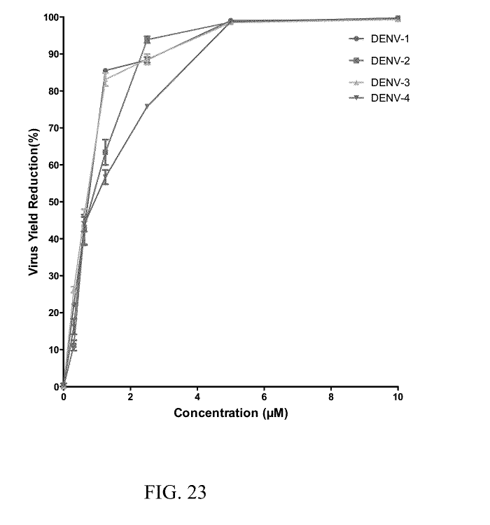

FIG. 23 shows the percent viral yield reduction the indicated DENV serotypes. In all cases the percent viral yield reduction is reduced at a concentration of at least about 1 μM and increases to 100% after the AR-12 concentration is more than about 4 μM. The virus yield reduction assay using q-RT-PCR was used to evaluate the in vitro anti-dengue virus activity of AR-12. Cells were treated with AR-12 at 1 h post virus infection and continuously treated up to 2 days post infection. The respective DENV RNA copy numbers (after 2 days post infection) were quantified using qRT-PCR. The percentages DENV yield inhibition was obtained by comparing against untreated controls maintained in parallel. Data from triplicate experiments were plotted using Graph Pad Prism Version 5 (Graph Pad Software Inc., San Diego, Calif.).

### REFERENCES

1. Taubenberger J K, Kash J C: Influenza virus evolution, host adaptation, and pandemic formation. Cell Host Microbe 2010, 7(6):440-451.
2. Molinari N A, Ortega-Sanchez I R, Messonnier M L, Thompson W W, Wortley P M, Weintraub E, Bridges C B: The annual impact of seasonal influenza in the US: measuring disease burden and costs. Vaccine 2007, 25(27):5086-5096.
3. Richard M, Schrauwen E J, de Graaf M, Bestebroer T M, Spronken M I, van Boheemen S, de Meulder D, Lexmond P, Linster M, Herfst S et al: Limited airborne transmission of H7N9 influenza A virus between ferrets. Nature 2013, 501(7468):560-563.
4. Russell C A, Fonville J M, Brown A E, Burke D F, Smith D L, James S L, Herfst S, van Boheemen S, Linster M, Schrauwen E J et al: The potential for respiratory droplet-transmissible A/H5N1 influenza virus to evolve in a mammalian host. Science 2012, 336(6088):1541-1547.
5. Herfst S, Schrauwen E J, Linster M, Chutinimitkul S, de Wit E, Munster V J, Sorrell E M, Bestebroer T M, Burke D F, Smith D J et al: Airborne transmission of influenza A/H5N1 virus between ferrets. Science 2012, 336(6088):1534-1541.
6. Xu R, de Vries R P, Zhu X, Nycholat C M, McBride R, Yu W, Paulson J C, Wilson I A: Preferential recognition of avian-like receptors in human influenza A H7N9 viruses. Science 2013, 342(6163):1230-1235.
7. Imai M, Watanabe T, Hatta M, Das S C, Ozawa M, Shinya K, Zhong G, Hanson A, Katsura H, Watanabe S et al: Experimental adaptation of an influenza H5 HA confers respiratory droplet transmission to a reassortant H5 HA/H1N1 virus in ferrets. Nature 2012, 486(7403):420-428.
8. Loregian A, Mercorelli B, Nannetti G, Compagnin C, Palu G: Antiviral strategies against influenza virus: towards new therapeutic approaches. Cell Mol Life Sci 2014, 71(19):3659-3683.
9. Chiu H C, Kulp S K, Soni S, Wang D, Gunn J S, Schlesinger L S, Chen C S: Eradication of intracellular Salmonella enterica serovar Typhimurium with a small-molecule, host cell-directed agent. Antimicrob Agents Chemother 2009, 53(12):5236-5244.
10. Gao M, Yeh P Y, Lu Y S, Hsu C H, Chen K F, Lee W C, Feng W C, Chen C S, Kuo M L, Cheng A L: OSU-03012, a novel celecoxib derivative, induces reactive oxygen species-related autophagy in hepatocellular carcinoma. Cancer Res 2008, 68(22):9348-9357.
11. Booth L, Cazanave S C, Hamed H A, Yacoub A, Ogretmen B, Chen C S, Grant S, Dent P: OSU-03012 suppresses GRP78/BiP expression that causes PERK-dependent increases in tumor cell killing. Cancer Biol Ther 2012, 13(4):224-236.
12. Chabrier-Rosello Y, Gerik K J, Koselny K, DiDone L, Lodge J K, Krysan D J: Cryptococcus neoformans Phosphoinositide-Dependent Kinase 1 (PDK1) Ortholog Is Required for Stress Tolerance and Survival in Murine Phagocytes. Eukaryot Cell 2013, 12(1):12-22.
13. Liu J, Qin C K, Lv W, Zhao Q, Qin C Y: OSU-03012, a non-cox inhibiting celecoxib derivative, induces apoptosis of human esophageal carcinoma cells through a p53/Bax/cytochrome c/caspase-9-dependent pathway. Anti-Cancer Drug 2013, 24(7):690-698.
14. West N W, Garcia-Vargas A, Chalfant C E, Park M A: OSU-03012 sensitizes breast cancers to lapatinib-induced cell killing: a role for Nck1 but not Nck2. BMC Cancer 2013, 13:256.
15. Silva A, Wang J, Lomahan S, Tran T A, Grenlin L, Suganami A, Tamura Y, Ikegaki N: Aurora kinase A is a possible target of OSU03012 to destabilize MYC family proteins. Oncol Rep 2014, 32(3):901-905.
16. Booth L, Roberts J L, Cruickshanks N, Grant S, Poklepovic A, Dent P: Regulation of OSU-03012 toxicity by ER stress proteins and ER stress-inducing drugs. Mol Cancer Ther 2014, 13(10):2384-2398.
17. Gannage M, Dormann D, Albrecht R, Dengjel J, Torossi T, Ramer P C, Lee M, Strowig T, Arrey F, Conenello G et al: Matrix Protein 2 of Influenza A Virus Blocks Autophagosome Fusion with Lysosomes. Cell Host Microbe 2009, 6(4):367-380.
18. Zhirnov O P, Klenk H D: Influenza A virus proteins NS1 and hemagglutinin along with M2 are involved in stimulation of autophagy in infected cells. J Virol 2013, 87(24):13107-13114.
19. Zhang R, Chi X, Wang S, Qi B, Yu X, Chen J L: The regulation of autophagy by influenza A virus. Biomed Res Int 2014, 2014:498083.
20. Aeffner F, Woods P S, Davis I C: Activation of A1-adenosine receptors promotes leukocyte recruitment to the lung and attenuates acute lung injury in mice infected with influenza A/WSN/33 (H1N1) virus. J Virol 2014, 88(17):10214-10227.
21. Aeffner F, Abdulrahman B, Hickman-Davis J M, Janssen P M, Amer A, Bedwell D M, Sorscher E J, Davis I C: Heterozygosity for the F508del mutation in the cystic fibrosis transmembrane conductance regulator anion channel attenuates influenza severity. J Infect Dis 2013, 208(5):780-789.
22. Arndt V et al., To be, or not to be—molecular chaperones in protein degradation. Cell Mol Life Sci. 2007; 64: 2525-2541.
23. Bukau B et al., Molecular chaperones and protein quality control. Cell 2006; 125(3):443-451.
24. Booth L et al. OSU-03012 suppresses GRP78/BiP expression that causes PERK-dependent increases in tumor cell killing. Cancer Biol. Ther. 2012; 13(4):224-236.
25. Booth L. et al. Regulation of OSU-03012 Toxicity by ER Stress Proteins and ER Stress-Inducing Drugs. Molecular Cancer Therapeutics. 2014; 13(10): 2384-2398.
26. Booth L. et al., GRP78/BiP/HSPA5/Dna K is a universal therapeutic target for human disease. Journal of Cellular Physiology 2014; http://onlinelibrary.wiley.com/doi/10.1002/jcp.24919/abstract
27. Chakrabarti A., A Review of the Mammalian Unfolded Protein Response. Biotechnol Bioeng 2011; 108(12): 2777-2793
28. Chase G et al., Hsp90 inhibitors reduce influenza virus replication in cell culture. Virology 2008; 377(2):431-439.
29. Connor J H et al., Antiviral activity and RNA polymerase degradation following Hsp90 inhibition in a range of negative strand viruses. Virology 2007; 362:109-119
30. Das I et al., Heat Shock Protein 90 Positively Regulates Chikungunya Virus Replication by Stabilizing Viral Non-Structural Protein nsP2 during Infection. Plos One 2014; 9 (6), e100531
31. Delang L., et al., Mutations in the chikungunya virus non-structural proteins cause resistance to favipiravir (T-705), a broad-spectrum antiviral, J Antimicrob Chemother 2014; 69: 2770-2784.
32. Dowall S D, et al. Elucidating variations in the nucleotide sequence of Ebola virus associated with increasing pathogenicity. Genome Biology 2014, 15:540-552.
33. Dutta D et al., The molecular chaperone heat shock protein-90 positively regulates rotavirus infection. Virology 2009, 391(2):325-333.
34. Evers D L et al, 17-allylamino-17-(demethoxy) geldanamycin (17-AAG) is a potent and effective inhibitor of human cytomegalovirus replication in primary fibroblast cells. Arch Virol 2012; 157(10):1971-1974.
35. Gao et al. Inhibition of HSP90 attenuates porcine reproductive and respiratory syndrome virus production in vitro Virology Journal 2014, 11:17
36. Geller R et al., Evolutionary constraints on chaperone-mediated folding provide an antiviral approach refractory to development of drug resistance. Genes & development 2007; 21: 195-205.
37. Geller R et al., Broad action of Hsp90 as a host chaperone required for viral replication. Biochim Biophys Acta (BBA)-Mol Cell Res 2012; 1823(3):698-706.
38. Geller H et al., Hsp90 Inhibitors Exhibit Resistance-Free Antiviral Activity against Respiratory Syncytial Virus PLOS ONE 2013; 8(2): e56762
39. Gire S K, et al. Genomic surveillance elucidates Ebola virus origin and transmission during the 2014 outbreak. Science 2014, 345(6202) 1369-1372
40. Hartl F U et al., Molecular chaperones in protein folding and proteostasis. Nature 2011; 475(7356):324-332.
41. He B. Viruses, endoplasmic reticulum stress, and interferon responses. Cell Death and Differentiation 2006; 13: 393-403.
42. Huang K L et al. Involvement of GRP78 in inhibition of HBV secretion by Boehmeria nivea extract in human HepG2 2.2.15 cells. J Viral Hep. 2009; 16: 367-375.
43. Hung J J et al. Molecular chaperone Hsp90 is important for vaccinia virus growth in cells. J Virol 2002; 76(3):1379-1390.
44. Kondratowicz et al., T-cell immunoglobulin and mucin domain 1 (TIM-1) is a receptor for Zaire Ebolavirus and Lake Victoria Marburgvirus. PNAS 2011; 109(20); 8426-8431
45. Krishnan A. et al., Niemann-Pick C1 (NPC1)/NPC1-like1 Chimeras Define Sequences Critical for NPC1's Function as a Filovirus Entry Receptor. Viruses 2012; 4; 2471-2484
46. Kugelman J R., et al. Evaluation of the Potential Impact of Ebola Virus Genomic Drift on the Efficacy of Sequence-Based Candidate Therapeutics. mBio; 2015 6(1):1 e02227-14
47. Lahaye X, et al. Hsp70 Protein Positively Regulates Rabies Virus Infection. Journal of Virology 2012; 86(9); 4743-4751
48. Lee A S. The ER chaperone and signaling regulator GRP78/BiP as a monitor of endoplasmic reticulum stress. Methods. 2005: 35(4): 373-381.
49. Li Y H et al., Geldanamycin, a ligand of heat shock protein 90, inhibits the replication of herpes simplex virus type 1 in vitro. Antimicrob Agents Chemother 2004; 48(3):867-872.
50. Ma Y et al., ER chaperone functions during normal and stress conditions. J Chem Neuroanat. 2004; 28: 51-65.
51. Mayer M P. Recruitment of Hsp70 chaperones: a crucial part of viral survival strategies. Rev. Physiol. Biochem. Pharmacology. 2005: 153; 1-46.
52. McClellan A J et al., Protein quality control: chaperones culling corrupt conformations. Nat Cell Biol. 2005; 7: 736-741.
53. Nakagawa S et al., Hsp90 inhibitors suppress HCV replication in replicon cells and humanized liver mice. Biochem Biophys Res Commun 2007; 353(4):882-888.
54. Neckers L. Molecular Chaperones in Pathogen Virulence: Emerging New Targets for Therapy. Cell Host Microbe. 2008; 4(6): 519-527
55. Paingankar M S, et al., dentification of chikungunya virus interacting proteins in mammalian cells. J. Bioscience 2014; 39; 389-399
56. Rathore et al., Chikungunya virus nsP3 & nsP4 interacts with HSP-90 to promote virus replication: HSP-90 inhibitors reduce CHIKV infection and inflammation in vivo. Antiviral Research. 2014; 103; 7-16
57. Reid P et al. HSPA5 is an essential host factor for Ebola virus infection. Antiviral Research. 2014; 109: 171-174.
58. Smith D R et al., Inhibition of heat-shock protein 90 reduces Ebola virus replication. Antiviral Res 2010; 87(2):187-194.
59. Wati S et al. Dengue virus infection indices up regulation of GRP78, which acts to chaperone viral antigen production. 2009: Journal of Virology; 83(24): 12871-12880.
60. Welch W J, How cells respond to stress. Sci Am. 1993: 268: 56-64
61. Xiao A., et al Viral interaction with molecular chaperones: role in regulating viral infection. Arch Virol 2010; 155:1021-1031
62. Young J C et al., Pathways of chaperone mediated protein folding in the cytosol. Nat Rev Mol Cell Biol 2004; 5(10):781-791.
63. Yuan W et al., Inhibition of infectious bursal disease virus infection by artificial microRNAs targeting chicken heat-shock protein 90. J Gen Virol 2012; 93:876-879.
64. Murray et al., Medical Microbiology 7^{th} Edition, Elsevier 2013.
65. Vallejo et al. Veterinary Research 2014, 45:15.

Not every element described herein is required. Indeed, a person of skill in the art will find numerous additional uses of and variations to the methods described herein, which the inventors intend to be limited only by the claims. All references cited herein are incorporated by reference in their entirety.

What is claimed as new and desired to be protected by Letters Patent of the United States is:

1. A method of inhibiting viral protein production in a host infected with a virus, comprising:
administering AR-12 to the host wherein viral protein production is reduced by at least about 50% compared to an untreated host.

2. The method of claim 1, wherein the viral protein is produced by influenza.

3. The method of claim 2, wherein the influenza virus is influenza A.

4. The method of claim 2, wherein the viral protein is structural protein.

5. The method of claim 2, wherein the viral protein is non-structural protein.

6. A method of inhibiting viral replication in a host infected with a virus, comprising:
comprising administering AR-12 to the host in an amount sufficient to reduce viral replication in the host by at least about 50%.

7. The method of claim 6, wherein the virus is selected from the group consisting of a Group I virus, Group IV virus, Group V virus, and Group VII virus.

8. The method of claim 6, wherein the virus is selected from the group consisting of influenza, HIV, HIV-1, HIV-2, drug-resistant HIV, Junin virus, Chikungunya virus, Yellow Fever virus, Dengue virus, Pichinde virus, Lassa virus, adenovirus, Measles virus, Punta Toro virus, Respiratory Syncytial virus, Rift Valley virus, RHDV, SARS coronavirus, Tacaribe virus, and West Nile virus.

9. The method of claim 6, wherein AR-12 is provided to the host in an amount sufficient to achieve a blood, tissue, or organ concentration from about 0.1 μM to about 7 μM.

10. A method of reducing viral infection in a host infected with Chikungunya virus comprising administering AR-12 to the host in an amount sufficient a blood, tissue, or organ concentration from of about 1 μM to 100 μM.

11. The method of claim 10, wherein the virus is a favipiravir-resistant strain of Chikungunya virus.

12. A method of reducing viral infection in a host infected with Junin virus comprising administering AR-12 to the host in an amount sufficient to achieve a blood, tissue, or organ concentration of about 0.15 μM to 0.55 μM.

13. A method of inhibiting retroviral replication in a host infected with a retrovirus, comprising administering AR-12 to the host in an amount sufficient to achieve a blood, tissue, or organ concentration of about 0.1 μM to about 20 μM.

14. The method of claim 13, wherein AR-12 is administered to the host in an amount sufficient to achieve a blood or tissue concentration of about 0.30 μM.

15. The method of claim 13, further comprising administering at least a second compound to the host.

16. The method of claim 15, wherein the second compound is selected from the group consisting of tipranavir, indinavir, atazanavir, saquinavir, lopinavir, ritonavir, darunavir, atazanavir, nelfinavir, emtricitabine, lamivudine {3TC}, zidovudine {AZT}, didanosine, tenofovir, stavudine, abacavir, rilpivirine, etravirine, nevirapine, delavirdine, efavirenz, T-20, maraviroc, raltegravir, and dolutegravir.

17. A method of inhibiting replication of HIV DR in a host infected with HIV DR, comprising administering AR-12 to the host in an amount sufficient to achieve a blood, tissue, or organ concentration of at least about 0.30 μM.

18. A method of increasing the survival rate of a host having viral-induced fulminant hepatic failure, comprising administering AR-12 to the host in an amount of at least about 25 mg/kg.

19. The method of claim 18, wherein AR-12 is administered to the host in amount of at least four doses of 25 mg/kg.

20. The method of claim 19, wherein the survival rate of the host is increased by at least about 20%.

21. A method of reducing Dengue virus infection in a host infected with Dengue virus, comprising administering AR-12 to the host in an amount sufficient to achieve a blood, tissue, or organ concentration of at least about 1 μM.

22. The method of claim 21, wherein the blood, tissue, or organ concentration is at least about 4 μM.
